(19) United States
(12) Patent Application Publication
(10) Pub. No.: US 2018/0049731 A1
(43) Pub. Date: Feb. 22, 2018

(54) CLOSING DEVICE FOR TISSUE OPENINGS

(71) Applicant: Muffin Incorporated, West Lafayette, IN (US)

(72) Inventors: Gregory James Hardy, Winston Salem, NC (US); Shaun Davis Gittard, Winston-Salem, NC (US); John C. Sigmon, JR., Winston-Salem, NC (US); William J. Havel, West Lafayette, IN (US); Jeremy T. Newkirk, West Lafayette, IN (US); Neal E. Fearnot, West Lafayette, IN (US); Rita Hadley, Otterbein, IN (US); Danny A. Sherwinter, Brooklyn, NY (US); Vihar C. Surti, Winston-Salem, NC (US); Tyler E. McLawhorn, Winston-Salem, NC (US)

(73) Assignee: Muffin Incorporated, West Lafayette, IN (US)

(21) Appl. No.: 15/800,437

(22) Filed: Nov. 1, 2017

Related U.S. Application Data
(63) Continuation-in-part of application No. 14/748,992, filed on Jun. 24, 2015.
Application No. 14/748,992 is a continuation-in-part of application No. PCT/US2015/037378, filed on Jun. 24, 2015.
Application No. PCT/US2015/037378 is a continuation-in-part of application No. 15/099,068, filed on Apr. 14, 2016.
Application No. 15/099,068 is a continuation of application No. 13/096,433, filed on Apr. 28, 2011, now abandoned.
Application No. 13/096,433 is a continuation-in-part of application No. 15/602,457, filed on May 23, 2017.
(60) Provisional application No. 62/018,986, filed on Jun. 30, 2014.
(60) Provisional application No. 62/018,986, filed on Jun. 30, 2014.
(60) Provisional application No. 61/343,435, filed on Apr. 29, 2010.
(60) Provisional application No. 61/379,243, filed on Sep. 1, 2010.

(51) Int. Cl. A61B 17/00; A61F 2/00; A61B 17/04

(52) U.S. Cl. CPC A61B 17/0057; A61B 2017/00619; A61B 2017/0496; A61B 2017/00876; A61B 2017/00867; A61B 2017/00676; A61B 2017/00637; A61B 2017/00623; A61B 17/0487; A61B 2017/00615; A61B 2017/00606; A61B 2017/00601; A61B 2017/00597; A61B 2017/00592; A61B 2017/00579; A61B 2017/00575; A61B 2017/00004; A61F 2002/0072; A61F 2/0063

(57) ABSTRACT

There are shown and described embodiments of closure devices and systems for closing holes in tissue such as those caused unintentionally (e.g. hernia) or intentionally (e.g. openings in the right atrial appendage to access cardiac tissue). Such devices and systems include one or more mesh elements or anchors. In particular embodiments, a closure device includes first and second mesh closure members and a tether or stem connecting them. Embodiments of a delivery device for such closure devices are also described.

This application is a continuation-in-part and claims the benefit of priority of U.S. application Ser. No. 14/748,992 and PCT US2015/037378, both filed on Jun. 24, 2015, which each claim the benefit of priority of U.S. Provisional Application Ser. No. 62/018,986, entitled “Expandable Mesh with Locking Feature,” filed Jun. 30, 2014. This application is also a continuation-in-part and claims the benefit of priority of U.S. application Ser. No. 15/099,068, entitled “Systems and Methods for Facilitating Closure of Bodily Openings,” filed Apr. 14, 2016, which is a continuation of U.S. application Ser. No. 13/096,433 filed Apr. 28, 2011, which claims the benefit of priority of U.S. Provisional Application Ser. No. 61/343,435, filed Apr. 29, 2010, and 61/379,243, filed Sep. 1, 2010. This application is also a continuation-in-part and claims the benefit of priority of U.S. application Ser. No. 15/602,457, entitled “Closing Device for Tissue Openings,” filed May 23, 2017. All of the above-listed applications are incorporated herein by reference in their entireties.

### BACKGROUND

The present embodiments relate generally to medical devices, and more particularly, to an expandable mesh that may be used in a variety of procedures. For example, the present application discloses structure and methods for closing internal tissue openings, including a closure with two connected mesh structures that may be used to close a minimally-invasive surgical opening in the heart or other organ.

There are many instances in which it may be desirable to deliver an expandable mesh into a human or animal body. By way of example, and without limitation, such expandable meshes may be used to treat perforations in tissue or bodily walls that are formed intentionally or unintentionally.

Perforations in tissue or bodily walls may be formed intentionally or unintentionally. For example, an unintentional abdominal hernia may be formed in the abdominal wall due to any of a number of reasons, or intentional perforations may be formed, for example, during surgical procedures such as translumenal procedures. Attempts to seal perforations have been attempted by coupling a graft member to tissue. The graft material may completely overlap with the perforation, and the edges of the graft material may at least partially overlap with tissue surrounding the perforation. The graft material then may be secured to the surrounding tissue in an attempt to effectively cover and seal the perforation. In order to secure the graft material to the surrounding tissue, sutures commonly are threaded through the surrounding tissue. However, such manual suturing techniques may be time consuming and/or difficult to perform.

There is a hernia repair method commonly referred to as a “mesh plug” or “plug and patch” repair technique, in which a surgeon uses a mesh plug to fill the perforation. Potential advantages include fewer sutures and less tissue dissection. However, a mesh plug alone may not effectively cover the entire area of the perforation, or alternatively, the mesh plug may shrink, become loose, or poke into adjacent tissue.

Minimally-invasive surgical procedures have been developed for placement of medical devices inside a patient or other therapeutic or diagnostic purposes, as a way of reducing trauma to a patient. In such procedures, holes are made and accessed by catheters or similar devices, and treatment devices are passed through the catheters to the site of interest. When the procedure or a part of it is concluded, the access catheter is removed and the hole repaired.

Devices and methods have been described for suturing such holes to close them. However, such devices and methods are commonly very difficult to use in such limited spaces. To avoid sutures, devices have been developed to plug or cover such holes, to allow the hole to heal naturally or incorporate some or all of such plugs into the tissue. Such items have been effective, but may be difficult to place, particularly when both sides of a hole must be closed.

### SUMMARY

The present embodiments provide a system for facilitating closure of a bodily opening. In one embodiment, the system comprises an anchor having a deployed state dimensioned for engaging tissue surrounding the opening, a first tether coupled to the anchor and extending proximally therefrom, and may include a graft member comprising a first bore disposed therein. The anchor may comprise a width that is larger than a width of the opening such that the anchor is disposed securely within or distal to the opening. The first tether is dimensioned to be disposed through the graft member, such that the graft member can be advanced distally over the first tether. The graft member may be secured to the anchor.

In one embodiment, the anchor comprises a plug of material including a plurality of filaments. The plug of material may comprise a diamond shape as initially prepared, in a pre-deployment state it can be positioned in a catheter or other delivery tube and take on a cylindrical shape, and have a deployed state having an increased width relative to the pre-deployment state. As the anchor emerges from the delivery tube, it will relax and begin to take the initial diamond shape, and will flatten as it is pulled against a tissue opening, as will be discussed further below. In an alternative embodiment, the anchor comprises a plurality of deployable members that are biased radially outward in the deployed state.

Particular embodiments provide an expandable mesh comprising a first coupling element, a second coupling element, and an intermediate portion disposed between the first coupling element and the second coupling element. Proximal retraction of the first coupling element relative to the second coupling element causes the intermediate portion to flare out to an enlarged width. Distal extension of the second element while maintaining position of the first coupling element can also be used to accomplish flaring to an enlarged width.

In one embodiment, the first coupling element comprises a first tube and the second coupling element comprises a second tube. In one example, the first tube, the second tube, and the intermediate portion each originate from the same mesh material. In one example the intermediate portion comprises untreated mesh material, and the first and second tubes are formed from treating the mesh material in a manner that maintains a tubular shape of the first and second tubes. At least one of the first tube or the second tube may be formed by melting or heat-shrinking the mesh material.

The expandable mesh may comprise a delivery state in which the first and second tubes lack an axial overlap, and further may comprise an expanded state in which the first and second tubes at least partially axially overlap. In one embodiment, a distal end of the first tube transitions into a first end of the intermediate portion, and a second end of the intermediate portion transitions into a distal end of the second tube.

The expandable mesh may comprise first and second ends. In one example, the expandable mesh may have a first state in which the first end is positioned proximal to the second end, and an everted second state in which the second end is positioned proximal to the first end.

The first and second tubes may be dimensioned to be secured together using a friction fit when the first tube is proximally retracted relative to the second tube. In one embodiment, one of the first and second tubes comprises a constant diameter along its length, while the other of the first and second tubes comprises a tapered shape. In an alternative embodiment, both the first and second tubes comprise tapered shapes, wherein the first tube is dimensioned to be disposed at least partially within the second tube when the first tube is proximally retracted relative to the second tube.

A system may be used with the expandable mesh. The system may comprise a first tether secured to the first coupling element, wherein proximal retraction of the first tether causes proximal retraction of the first coupling element relative to the second coupling element. Further, the system may comprise a graft material having a first bore formed therein, dimensioned for advancement over the first tether to permit the graft material to be advanced relative to the first coupling element.

The system may comprise a second tether coupled to the anchor, where the first tether is disposed through a first bore in the graft member and the second tether is disposed through a second bore in the graft member. The graft member may be advanced over the first and second tethers toward the anchor, and the first and second tethers are tied together to secure the graft member to the anchor.

Advantageously, an enhanced anchor and graft member attachment may be achieved to better treat the opening. For example, the anchor is capable of expanding to securely engage the opening. Additionally, the expanded anchor is secured to the graft member in a manner that may reduce the rate of migration of the anchor.

The devices disclosed as anchors or mesh elements can be part of systems or devices for closing tissue openings. For example, particular embodiments are disclosed of a closure device including a distal collapsible mesh element, a proximal collapsible mesh element, and a tether or stem that connects and is used to pull the two mesh elements together, sandwiching a hole to be sealed. The distal mesh element has two ends that are inverted into the mesh body interior. The mesh fibers at each of the ends are fused together or otherwise narrowed with a bonding or fusing operation such as shape-setting with heat, such as in the shape of tubes as noted above. Radiopaque markers (e.g. cylindrical markers) may be embedded in the fused ends. In particular embodiments, these ends are both inverted into the body of the distal mesh element. The distal end of the distal mesh element is used to anchor the distal end of the tether or stem. The proximal end of the distal mesh may be covered (internally or externally) with a material to seal and/or promote healing (e.g. small intestine submucosa [SIS]).

The proximal mesh element has a distal end that is inverted into the body of the proximal mesh element. The end is fused in an identical or similar manner to the ends of the distal mesh element and may also have a radiopaque marker. The distal end of the proximal mesh element may also be covered (internally or externally) with a material to seal and/or promote healing. In particular embodiments, the proximal end of the proximal mesh element is also fused or otherwise narrowed and incorporates a radiopaque marker, but is not inverted, as it is then able to fit into a relatively smaller delivery catheter or tube. It will be understood that the proximal end could be inverted in some embodiments. The proximal fused or narrowed end serves as an eventual conduit for the tether.

The tether has a distal end, which may be enlarged (e.g. with a bead, node or knot) and may be fixed to the distal end of the distal mesh element, has a cross-sectional enlargement (e.g. a bead, node or knot) at or near its proximal end. The tether also has a loop feature in particular embodiments that is a part of or adjacent to that proximal enlargement to allow attachment to a trigger or control line. Such a line may be a wire (e.g. of biocompatible metal), filament (e.g. a suture) or other line of a material that can be effectively used for pulling or controlling parts of the device. The line is used to pull the tether through the proximal end of the proximal mesh element as a delivery tool or device pushes and compresses the proximal mesh element. The proximal enlargement is pulled through the fused proximal end of the proximal mesh element and provides a lock or stop for the proximal mesh element once tension on the tether is released.

The delivery tool delivers the mesh elements in a stacked manner. The closure device is stacked within the tool with the distal mesh element residing in a distal peel-away catheter (e.g. 14 French), and the proximal mesh element residing in a sheath (e.g. 12 French). An access sheath, e.g. for pericardial access, having an anchoring balloon is (or has previously been) placed through a tissue hole to be closed. When the tool is inserted through the access sheath, which is anchored by its balloon on the distal side of the hole, the peel-away catheter is removed so that the distal mesh element sits within the sheath through the hole and is pushed by the sheath holding the proximal mesh element. The distal mesh element is pushed out of the sheath through the hole to a site distal to the hole to be sealed. The tether is then slightly retracted which serves to compress the distal mesh element against the distal end of the access sheath. The distal mesh element is thereby expanded. After deflating the balloon, the two sheaths are pulled back through the hole, which pulls the distal mesh element against the tissue and seals the hole.

In embodiments in which one or both sheaths include a fluid pathway, a contrast medium may be moved through that pathway to the site to allow visualization (e.g. by fluoroscopy) so as to check the seal created by the distal mesh element. After confirming a seal, and confirming that the sheath tips are on the proximal side of the hole, the proximal mesh element is pushed out of its sheath with a smaller inner tube or sheath (e.g. 9 French), alone or with further retraction of the sheath that held the proximal mesh element. Tension is maintained on the tether or stem via the control line throughout to ensure that the distal mesh element maintains a seal of the hole. The inner tube or sheath continues to push the proximal end of the proximal mesh element so as to advance that proximal end over the control line and ultimately over the proximal enlargement on the tether or stem, locking the mesh elements together. A final contrast injection can be made to confirm the seal of the hole, and an end of the control line is released to allow it to unloop from the tether or stem end.

In particular embodiments, the tether or stem may have multiple enlargements (e.g. knots, beads or nodes) to allow variable amounts of tightening of the mesh elements together. The proximal end of the proximal mesh element may have reliefs cut into it to allow some expansion as enlargement(s) of the tether or stem are pulled through that proximal end, and/or have a tapered hole to favor unidirectional movement of the tether or stem enlargement(s) through. Other gripping, attachment or reversion preventers or minimizers may be used, such as a barb, claw or corkscrew in the proximal end of the proximal mesh element to engage the tether or stem. A handle of the delivery device or tool may have one or more actuators or other mechanisms to promote performing deployment steps in the proper order and to minimize the chance of premature deployment or release of any component during the procedure.

As examples, a closure for an opening in tissue can include a first closure element, the first closure element having a first mesh enclosure, the first mesh enclosure having a first distal narrowed end and a second proximal narrowed end and a central volume. Each of the first and second ends are inverted so as to be within the central volume of the first mesh enclosure, and each of the first and second ends are surrounded by respective external surfaces of the first mesh enclosure. A second closure element has a second mesh enclosure with a third distal narrowed end and a fourth proximal narrowed end and a central volume. The third end is inverted so as to be within the central volume of the second mesh enclosure, and each of the third and fourth ends are surrounded by respective external surfaces of the second mesh enclosure. A tether joins the first and second closure elements in an initial configuration prior to delivery of the closure elements to the opening. The tether has first and second enlarged ends, wherein the tether extends through the first, second and third narrowed ends so that the first enlarged end of the tether is outside the first closure element adjacent or engaging the first end and the second enlarged end of the tether is within the central volume of the second closure element. The first closure element is adapted to engage a distal side of the tissue having the opening, and the second closure element is adapted to engage a proximal side of the tissue, and the tether is adapted to pass through the opening.

The mesh for the closure elements and the material for the tether or stem are preferably bioresorbable. As the closure elements are formed or prepared, a heat-annealing or shape-set process may be performed on them so that even though compressed or otherwise fitted within a delivery device, the closure elements naturally expand when deployed from the delivery device.

A sheet of bioresorbable material (e.g. the graft, seal or healing material referred to herein) may be fixed to the first closure element adjacent or over the second narrowed end. Such a sheet can be fixed to an external or internal surface or portion of the first mesh enclosure, or to an internal or external surface or portion of the second mesh enclosure, or to both mesh enclosures. The enlarged ends of the tether can be or include a bead or a knot. A control line or wire is looped through the second enlarged end of the tether, and may pass through the fourth narrowed end. The closure device is preferably initially fitted within a delivery device. The ends of the first closure element may be aligned with each other, and/or the ends of the second closure element may be aligned with each other. In other embodiments, the ends of the first closure element may be laterally offset with respect to each other, or one of those ends may be larger in diameter than the other.

An example of a device for closing an opening in tissue can include a delivery device having a first peel-away catheter, a second tube within the first peel-away catheter, and a pusher tube within the second tube, along with a closure device as disclosed herein fitted within the delivery device. For instance, a first closure element may be within the first peel-away catheter and a second closure element within the second tube. A control line may be looped through the second enlarged end of the tether. A control cannula may extend through the pusher tube, with the control line extending through the control cannula. The control line can extend from the control cannula and returns to the control cannula from the second enlarged end of the tether, so that a free end of the control line is within the control cannula.

Unlike available systems which use a single mesh construct or container with two collapsible disks to form distinct regions, particular embodiments described herein use two independent mesh constructs linked by a suture or other filament. The mesh constructs do not share any mesh or surface area This provides several advantages. For example, only a suture with its small diameter, rather than a much larger expanding-diameter mesh portion, sits between the independent mesh constructs to tether them together for delivery. The small suture diameter means there is little or no distention of the hole to be closed. Further, if the hole is not straight (e.g., substantially perpendicular to the adjacent tissue surface(s)), but is instead diagonal or crooked with respect to adjacent surface(s), the suture will not tend to deform the hole or its opening(s). As another example, the independent meshes allow spacing between each to be adjusted, particularly when the meshes are flattened in use as described below. This allows for such embodiments to accommodate a range of thicknesses of tissue through which the hole extends. As another example, with two independent mesh constructs each can conform to their respective surfaces independently. If the opposing (e.g. inside and outside) tissue surfaces are not parallel or are angled with respect to each other, for example where the tissue through which the hole extends is of variable thickness near or around the hole, each mesh construct can conform to its respective tissue surface independently. Neither mesh pulls on the other in such a configuration, lessening the likelihood of damage to the tissue.

Whether denoted as a “plug,” “anchor,” “mesh,” “closure member” or similar term, what is intended is a device for closing a hole in tissue. Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIGS. 1-5 are side-sectional views illustrating exemplary method steps that may be used to facilitate closure of an opening using a system according to a first embodiment comprising an anchor and a graft member.

FIG. 6 is a side view of a mesh disposed over a first mandrel.

FIG. 7 is a side view of the mesh after formation of a first tube.

FIG. 8 illustrates eversion of a portion of the mesh, with the first tube depicted in a side view and other mesh material shown in a side-sectional view.

FIGS. 9-10 illustrate exemplary method steps, with first and second tubes depicted in a side view and other mesh material also shown in a side view.

FIG. 11 illustrates the mesh in a delivery state, with the mesh shown in a side view and an insertion tool shown in a side-sectional view.

FIG. 12 illustrates deployment of the mesh, with the first tube depicted in a side view, and the second tube, other mesh material and the insertion tool shown in side-sectional views.

FIGS. 13-14 illustrate advancement of a graft member over a first tether coupled to the mesh, with the first tube depicted in a side view, and the second tube, other mesh material and the insertion tool shown in side-sectional views.

FIGS. 15A-15C are side views of alternative first and second tube configurations.

FIG. 16 is a perspective view of an embodiment of a closure device described herein.

FIG. 17 is a side view of an embodiment of a mesh portion prior to being formed into a part of the embodiment of FIG. 16.

FIG. 18 is a side part-cross-sectional view of a portion of the embodiment of FIG. 16.

FIG. 19 is a side part-cross-sectional view of a portion of the embodiment of FIG. 16 with an alternative joining member.

FIG. 20 is a side part-cross sectional view of an alternative closure element that can be used in the embodiment of FIG. 16.

FIG. 21 is a side part-cross sectional view of an alternative closure element that can be used in the embodiment of FIG. 16.

FIG. 22 is a side part-cross sectional view of an alternative closure element that can be used in the embodiment of FIG. 16.

FIG. 23 is a side part-cross-sectional view of a delivery device with the embodiment of a closure device of FIG. 16 fitted within it, in an initial stage of insertion into a patient.

FIG. 24 is a view of the embodiment of FIG. 23 in a later stage of deployment compared to FIG. 23.

FIG. 25 is a view of the embodiment of FIG. 23 in a later stage of deployment compared to FIG. 24.

FIG. 26 is a view of the embodiment of FIG. 23 in a later stage of deployment compared to FIG. 25.

FIG. 27 is a view of the embodiment of FIG. 23 in a later stage of deployment compared to FIG. 26.

FIG. 28 is a schematic representation of a portion of the delivery device embodiment shown in FIG. 23.

FIG. 29 is a side part-cross-sectional view of a portion of the delivery device embodiment of FIG. 23 with additional structure.

FIG. 30 is a side part-cross-sectional view of a portion of the delivery device embodiment of FIG. 23 with additional structure.

### DETAILED DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, and alterations and modifications in the illustrated devices and methods, and further applications of the principles of the disclosure as illustrated therein are herein contemplated as would normally occur to one skilled in the art to which the disclosure relates.

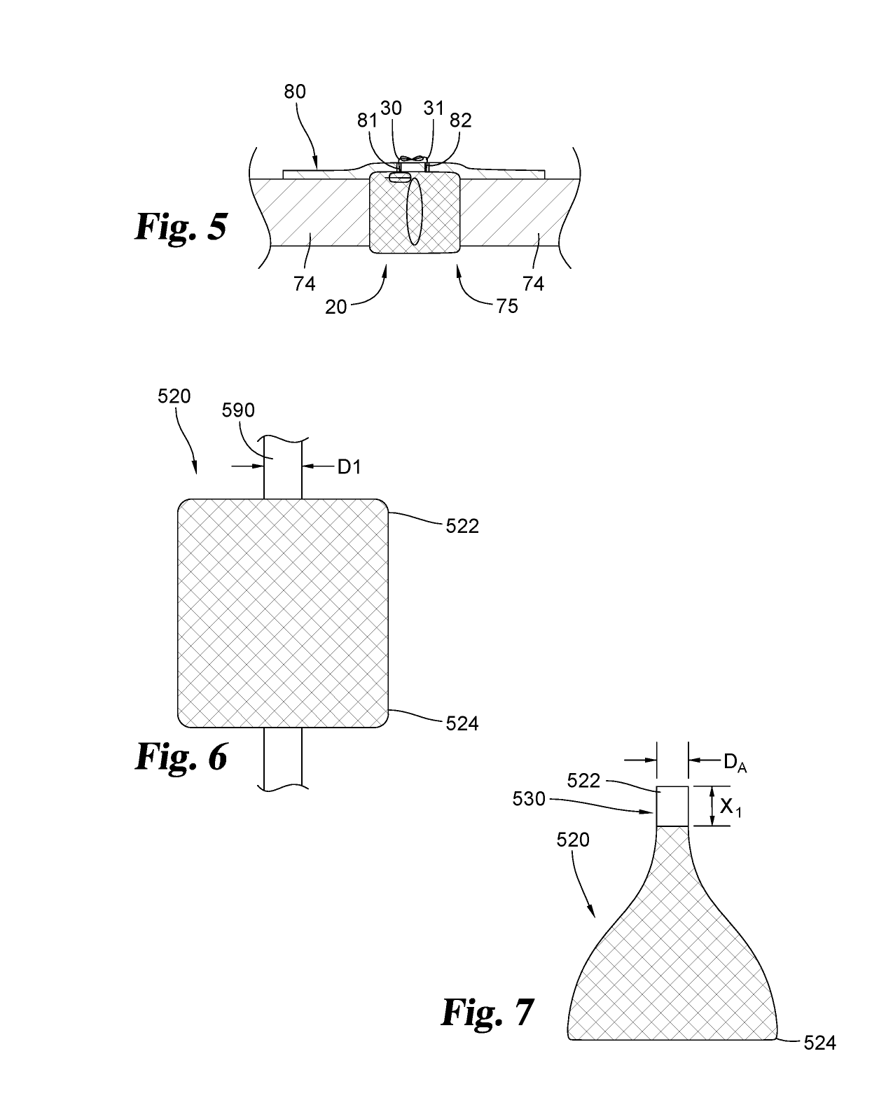

In the present application, the term “proximal” refers to a direction that is generally towards a physician during a medical procedure, while the term “distal” refers to a direction that is generally towards a target site within a patent's anatomy during a medical procedure. Thus, “proximal” and “distal” portions of a device or bodily region may depend on the point of entry for the procedure (e.g., percutaneously versus laparoscopically or endoscopically). Additionally, it is noted that when manufacturing a device according to one embodiment, an eversion step is performed whereby a portion that was originally a distal region of the device becomes a proximal region. For clarity, with respect to FIGS. 6-15C the region that is originally near a proximal end will be referred to as the first end, while the region that is originally near a distal end will be referred to as the second end.

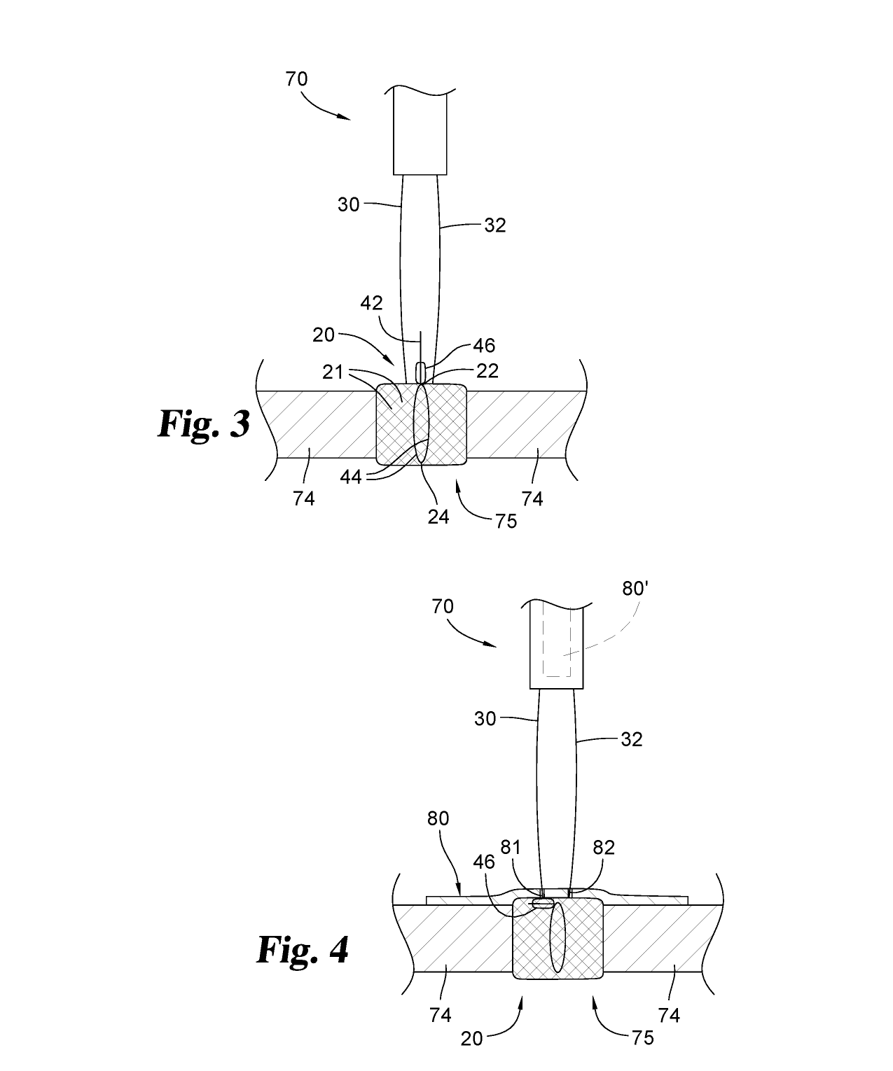

Referring now to FIGS. 1-5, systems and methods are described for facilitating closure of a bodily opening according to a first embodiment. The system comprises an anchor or closure member 20, which has pre-deployment and deployed states. In the pre-deployment state, the anchor 20 comprises a generally diamond shape having a proximal region 22, a distal region 24, and side regions 26 and 28, and further comprising a height h and a width w, as shown in FIG. 1. In this example, the anchor 20 may be formed of a plug of material, such as a plurality of filaments 21 that are woven together in a manner that allows compression of the filaments with respect to each other when a sufficient force is applied. In one embodiment, in the pre-deployment state the height h between the proximal and distal regions 22 and 24 may be about the same or greater than the width w between the side regions 26 and 28. Preferably, the width w of the anchor 20 in the pre-deployment state is greater than a width wₒ of an opening 75 formed in tissue 74. As will be explained further below, by oversizing the width w of the anchor 20 relative to the width wₒ of the opening 75, the anchor 20 may be frictionally held in place within or covering the opening 75. Moreover, the width w of the anchor 20 may be further increased in the deployed state using an actuator 40, as explained further in FIG. 3 below, to further enhance the frictional engagement with the tissue 74 surrounding the opening 75.

The anchor 20 can be fashioned from absorbable and non-absorbable mesh or biologic implant with or without spars of absorbable or non-absorbable material to help it retain its shape and anchorage. The mesh can be shaped like an umbrella or diamond. The deployed expanded shape can be maintained with suture material or a locking mechanism or through the inherent shape and orientation of the spars. In addition, the anchor 20 can be fashioned out of absorbable or non-absorbable spars or a metallic material (e.g., nitinol, stainless steel etc.) shaped as an umbrella, diamond or any shape that expands in diameter after deployment which can be deformed and compressed when placed into the deployment instrument and will then return to its expanded shape after deployment in the defect. In addition, legs of the anchor 20 can have small hooks or tines at the ends to catch on the surrounding tissue. The anchor can be made in multiple sizes for different depth and/or diameter defects.

The system further comprises a first tether 30 coupled to the anchor 20 and extending proximally therefrom, as shown in FIGS. 1-5. The first tether 30 is sized to be disposed through a first bore 81 in a graft member 80, thereby enabling distal advancement of the graft member 80 over the first tether 30 towards the anchor 20 after the anchor 20 has been deployed within the opening 75, as explained further in FIGS. 4-5 below. Optionally, a second tether 32 similarly may be coupled to the anchor 20, and disposed through a second bore 82 in the graft member 80. After distal advancement of the graft member 80 over the first and second tethers 30 and 32 toward the anchor 20, the first and second tethers 30 and 32 may be tied, thereby securing the graft member 80 in place relative to the anchor 20, as explained further in FIG. 5 below. In one example, the first and second tethers 30 and 32 each comprise monofilament sutures, though they can comprise single fibers or woven fibers, may be biodegradable, and have other suitable characteristics to perform the functions herein.

Optionally, the system may comprise an actuator 40 for laterally expanding the anchor 20 between the pre-deployment and the deployed states. In one example, the actuator 40 comprises a suture 42 having a distal region comprising a loop member 44, which may extend around the distal region 24 of the anchor 20 as shown in FIG. 1. The loop member 44 is coupled to a tensioning member 46 that is disposed adjacent to the proximal region 22 of the anchor 20. In use, the tensioning member 46 may be advanced distally over the suture 42 to reduce the overall diameter of the loop member 44, thereby moving the proximal region 22 towards the distal region 24 to reduce the height h, while increasing the width w between the side regions 26 and 28, as explained further in FIG. 3 below.

In the example shown, the opening 75 is a hernia located in the tissue 74 of the abdominal wall. While treatment of a hernia is shown for illustrative purposes, it will be apparent that the systems described herein may be used in a wide range of medical procedures, including open, laparoscopic, endoscopic, percutaneous and luminal procedures, and including but not limited to any exemplary procedures described herein.

The initial stages of hernia repair may be performed using various techniques, for example, an open technique, a laparoscopic technique, an endoscopic technique, or a percutaneous technique. In an open technique, an incision may be made in the patient, e.g. an abdominal or chest wall and the hernia may be repaired using generally known principles. In a laparoscopic technique, two or three smaller incisions may be made to access the surgical site. A laparoscope may be inserted into one incision, and surgical instruments may be inserted into the other incision(s). In an endoscopic technique, an endoscope may be advanced through a bodily lumen such as the alimentary canal, with an access hole being created through tissue to obtain access to the surgical site. One or more components, such as the insertion tool 70, may be advanced through a working lumen of the endoscope. The percutaneous approach is similar to the laparoscopic approach, but in the percutaneous approach the insertion tool 70 may be advanced directly through a patient's skin. In particular, with the components loaded, the insertion tool 70 is advanced directly through the abdominal skin, through the tissue 74, and may be advanced just distal to the opening 75 and into the peritoneum. In order to optimally visualize the insertion tool 70, a laparoscopic viewing device may be positioned in the peritoneum, or an endoscope may be translumenally advanced in proximity to the target site, as noted above. Alternatively, the insertion tool 70 and markers disposed thereon may be viewed using fluoroscopy of other suitable techniques. A transluminal approach, e.g. for the heart, may include accessing the vasculature of the patient and advancing tools through the vasculature to the surgical site, e.g. through a catheter.

After gaining access to the opening 75 using any of the above-referenced techniques, an insertion tool 70, such as a catheter or a needle, may be used to deliver one or more of the components of the system. If a needle is used, it may be an endoscopic ultrasound (EUS) or echogenic needle, such as the EchoTip® Ultrasound Needle, or the EchoTip® Ultra Endoscopic Ultrasound Needle, both manufactured by Cook Endoscopy of Winston-Salem, N.C.

The anchor 20 is disposed within a lumen of the insertion tool 70, as illustrated in the dashed delivery state 20′ of the anchor, shown in FIG. 1. The anchor may be advanced within the lumen of the insertion tool 70, e.g., using a stylet, and then is ejected from a distal end of the insertion tool 70. The anchor 20 assumes its pre-deployment state, as shown in FIG. 1. At this time, the first and second tethers 30 and 32, along with the suture 42 of the actuator 40, each extend proximally through the lumen of the insertion tool 70 for manipulation by a physician.

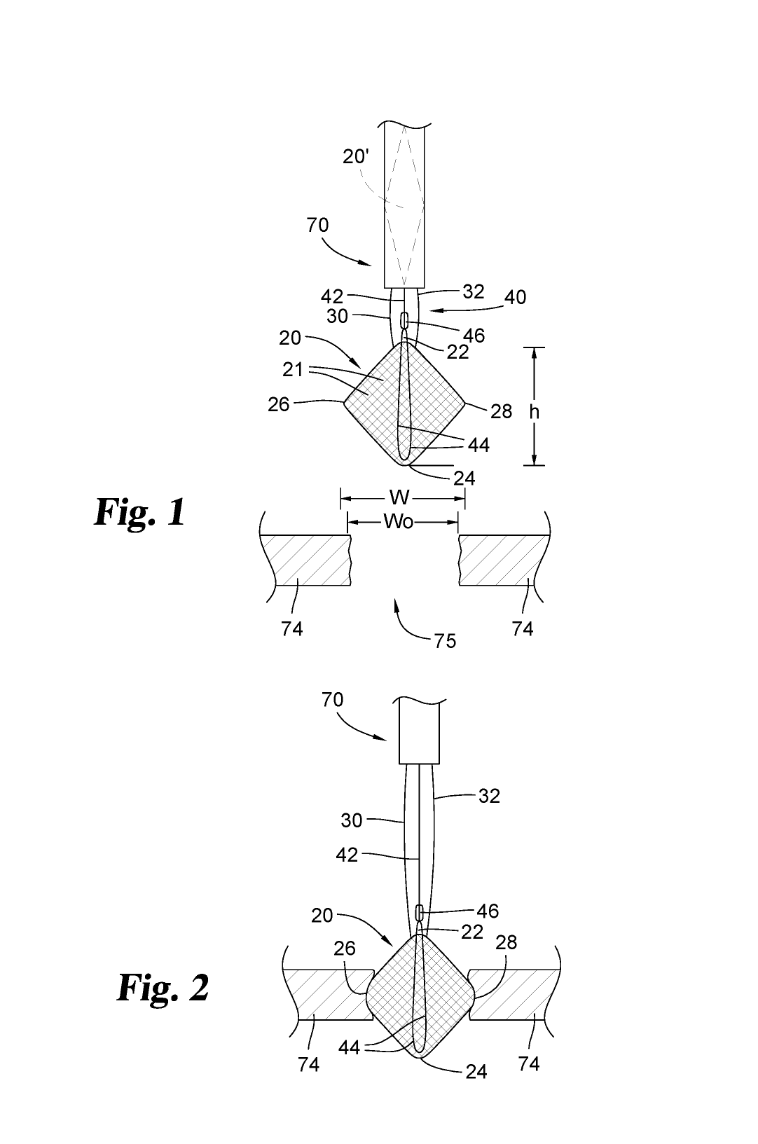

Referring to FIG. 2, the anchor 20 is advanced distally by a suitable device, such as a pusher tube, insertion tool, forceps or other grasping instrument. The anchor 20 is positioned within the opening 75, as shown in FIG. 2. Advantageously, the anchor 20 is diamond-shaped in the pre-deployment state, such that the distal region 24 is tapered to facilitate entry into the opening 75. Since the width w of the anchor 20 preferably is greater than the width wₒ of the opening 75 in the pre-deployment state, a force may be applied, e.g., using the pusher tube, insertion tool, forceps or other grasping instrument, to urge the anchor 20 in place so that at least the side regions 26 and 28 securely engage the tissue 74 surrounding the opening 75, as shown in FIG. 2. Alternatively, the anchor 20 may be deployed distal to the opening 75, in which case the anchor can assume a diameter larger than the opening 75 and provide anchoring functionality just distal to the tissue 74 with the same method steps otherwise being performed as shown herein.

Referring to FIG. 3, in a next step the actuator 40 is actuated to laterally expand the anchor 20, thereby further securing the anchor 20 within the opening 75 and/or distal to the opening 75. In particular, the tensioning member 46 is advanced distally over the suture 42 to reduce the overall diameter of the loop member 44, thereby moving the proximal region 22 towards the distal region 24 to reduce the height h, while increasing the width w between the side regions 26 and 28 of the anchor 20 to enhance a secure fit between the side regions 26 and 28 of the anchor 20 and the tissue 74 surrounding the opening 75. An increased width w of the anchor 20 in the deployed state of FIG. 3 may provide an increased frictional engagement with tissue disposed within the opening 75.

Preferably, the tensioning member 46 comprises a one-way movement feature, such as a cinching or ratcheting mechanism, to prevent proximal movement of the tensioning member 46 relative to the anchor 20 after deployment. Alternatively, the tensioning member 46 may comprise a rubber disc or beaded member, which may frictionally engage an exterior surface of the suture 42, but may be advanced distally over the suture 40 with a suitable external force. After actuating the actuator 40, the suture 42 may be cut by a suitable device, such as laparoscopic scissors, leaving the anchor 40 in place as shown in FIG. 3.

Referring to FIGS. 4-5, in a next step the graft member 80 may be advanced distally over the first and second tethers 30 and 32 towards the anchor 20. Properties of suitable graft members 80 are described in detail below. The graft member 80 comprises first and second bores 81 and 82, as noted above, which are sized to permit advancement of the graft member 80 over the first and second tethers 30 and 32, respectively.

In use, proximal ends of the first and second tethers 30 and 32 are disposed through the first and second bores 81 and 82 of the graft member 80 outside of the patient, and the graft member 80 is advanced distally relative to the first and second tethers 30 and 32. The graft member 80 may be delivered through the insertion tool 70, as depicted by the dashed lines of a graft member 80′ in the delivery state in FIG. 4. Alternatively, the graft member 80 may be delivered directly through a trocar, e.g., a 5 mm trocar. When ejected from the insertion tool 70 or the trocar, the graft member 80 then is positioned in place relative to the tissue 74 using a suitable grasping device, or a pusher tube or the insertion tool 70 itself, such that the graft member 80 is adjacent to the tissue 74 and covering the opening 75, as shown in FIG. 4.

In a next step, a suture tying device may be used to tie the first and second tethers 30 and 32 together in a manner that secures the graft member 80 adjacent to the tissue 74 and the anchor 20. By way of example, and without limitation, one suitable suture tying device is disclosed in U.S. patent application Ser. No. 12/125,525, filed May 22, 2008, the disclosure of which is hereby incorporated by reference in its entirety. Another suitable suture tying device is disclosed in U.S. patent application Ser. No. 12/191,001, filed Aug. 13, 2008, the disclosure of which is hereby incorporated by reference in its entirety. Upon completion of the tying procedure, the first and second tethers 30 and 32 may be cut by a suitable device, such as laparoscopic scissors, leaving the anchor 40 and the graft member 80 in place as shown in FIG. 5.

Advantageously, using the anchor 20, the first and second tethers 30 and 32, and the graft member 80 in combination, along with the techniques described, an enhanced anchor and graft member attachment may be achieved to comprehensively treat the opening 75. In this example, the anchor 20 is capable of expanding to fill the opening 75, potentially resulting in better tissue ingrowth and lower rates of recurrence. Moreover, the anchor 20 is secured within the opening 75 in an expanded, secure manner that may reduce anchor migration. Further, the coupling of the anchor 20 to the graft member 80 provides an enhanced seal relative to a plug alone, and the secure attachment of the anchor 20 to the graft member 80 may further reduce the rate of migration of the anchor 20.

The graft member 80 may comprise any suitable material for covering the opening 75 and substantially or entirely inhibiting the protrusion of abdominal matter. In one embodiment, the graft member 80 may comprise small intestinal submucosa (SIS), such as BIODESIGN® SURGISIS® Tissue Graft, available from Cook Biotech, Inc., West Lafayette, Ind., which provides smart tissue remodeling through its three-dimensional extracellular matrix (ECM) that is colonized by host tissue cells and blood vessels, and provides a scaffold for connective and epithelial tissue growth and differentiation along with the ECM components. The graft member 80 may be lyophilized, or may comprise a vacuum pressed graft that is not lyophilized. In one example, the graft member 80 would be a one to four layer lyophilized soft tissue graft made from any number of tissue engineered products. Reconstituted or naturally-derived collagenous materials can be used, and such materials that are at least bioresorbable will provide an advantage, with materials that are bioremodelable and promote cellular invasion and ingrowth providing particular advantage. Suitable bioremodelable materials can be provided by collagenous ECMs possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa. The graft member 80 may also comprise a composite of a biomaterial and a biodegradeable polymer. Additional details may be found in U.S. Pat. No. 6,206,931 to Cook et al., the disclosure of which is incorporated herein by reference in its entirety.

While the exemplary embodiments herein have illustrated the use of one or more systems for covering an opening 75 formed in the abdominal wall, the systems disclosed herein may be useful in many other procedures. Solely by way of example, the systems may be used to treat perforations in a visceral wall, such as the stomach wall. Further, the systems 20 may be used to secure a graft member to tissue for reconstructing local tissue, and the like.

An example of a mesh device usable as an anchor or closure device 20 is shown in FIG. 6. Referring to that figure, a mesh 520 having a first end 522 and a second end 524 is provided. The mesh 520 may be disposed over a first mandrel 590 having an outer diameter D₁, such that the first end 522 is initially disposed proximal to the second end 524. The mesh 520 can be fashioned from absorbable or non-absorbable mesh or biologic implant. By way of example, and without limitation, the mesh material may comprise polypropylene, polyethylene, glycolide/L-lactide copolymer, PTFE, nylon, polyurethane, PEEK, PLGA, PGA, polycaprolactone, carbothane, polydioxanone, or any copolymer of the aforementioned list.

Referring to FIG. 7, in a next step, the first end 522 of the mesh 520 is made to form a first coupling element 530. In this example, the first coupling element 530 is in the form of a first tube 530. However, it will be appreciated that the first coupling element 530 may take a form different than a tubular shape. For reference purposes below, the first coupling element 530 will be referenced as a first tube 530, although it is not intended to limit the shape of the first coupling element 530 to tubular form.

Since the first tube 530 is formed around the first mandrel 590, the first tube 530 comprises an inner diameter that is only slightly larger than the outer diameter D₁ of the first mandrel 590. Further, the first tube 30 comprises an outer diameter D_{A}, as shown in FIG. 7.

The first tube 30 is formed such that it comprises a length X₁, as shown in FIG. 7. In a presently preferred embodiment, the length X₁ is less than half of the overall length of the mesh 520, where the overall length is measured between the most proximal and distal endpoints of the mesh 520 in a flattened state of FIG. 6. Preferably, the length X₁ of the first tube 530 is between about 5.0 percent and about 33.0 percent of the overall length of the mesh 520 in the flattened state. In this manner, the length X₁ of the first tube 530 can most effectively cooperative with a subsequently formed second tube 40 and an intermediate portion 550, as will be explained further below.

In one exemplary technique, the first end 522 of the mesh 520 may be secured as the first tube 50 by melting or heat-shrinking the mesh material upon itself along the first end 522. In alternative embodiments, the first end 522 of the mesh 520 may be secured as the first tube 530 using a separate biocompatible adhesive, one or more biocompatible sutures, or other mechanisms that can maintain the structural integrity of the tubular shape for the purposes explained below.

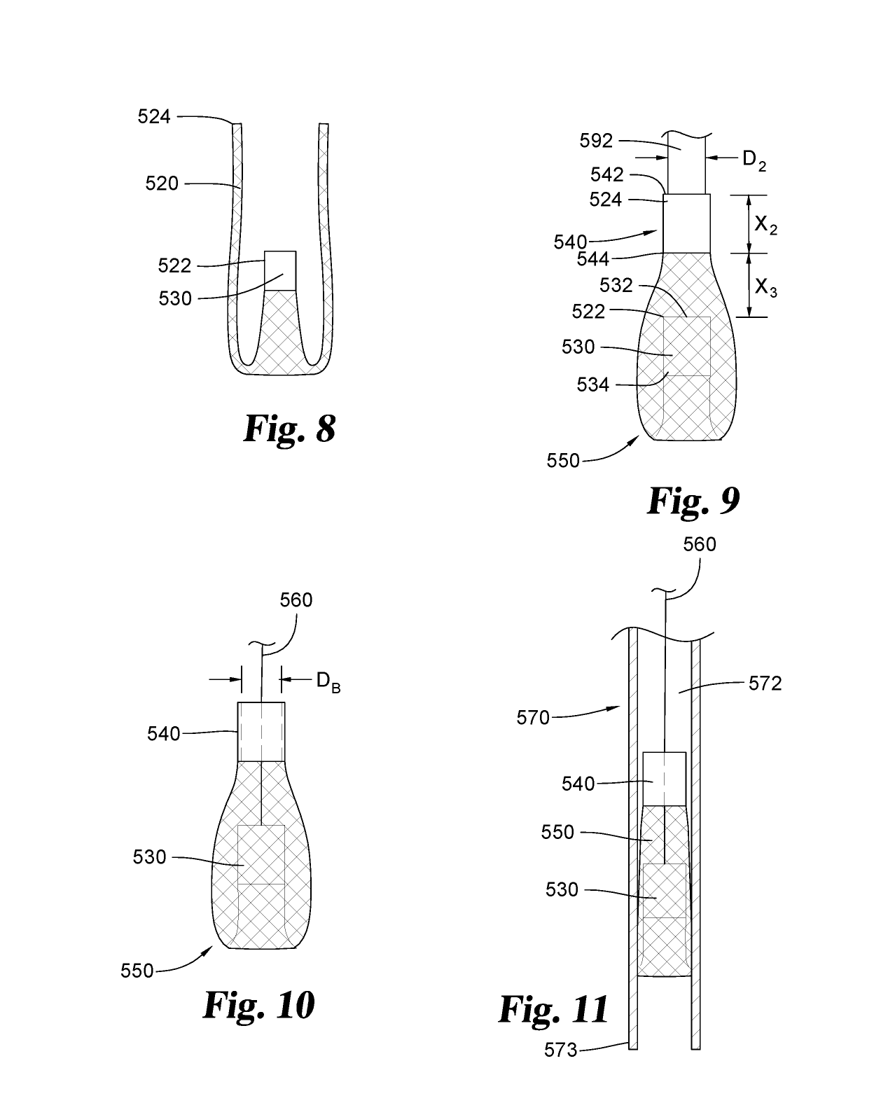

Referring now to FIG. 8, in a next step, the mesh 520 may be at least partially everted by moving the second end 524 proximally beyond the first end 522. In this manner, the second end 524 is brought radially over and around the first tube 50, as shown in FIG. 8. Therefore, in this eversion step, the second end 524 of the mesh 520 that was originally a distal region of the device has become a proximal region.

Referring to FIGS. 9-10, the second end 524 then is made into a second coupling element 540, for example, in a manner similar to which the first end 522 was made into the first coupling element 530. In this example, the second coupling element 540 is in the form of a second tube 540. However, it will be appreciated that the second coupling element 540 may take a form different than a tubular shape. For example, the second coupling element 540 may comprise a solid inner diameter, and still may engage an inner surface of the first coupling element 530 using a friction fit, as explained below. For reference purposes below, the second coupling element 540 will be referenced as a second tube 540, although it is not intended to limit the shape of the second coupling element 540 to tubular form.

In one embodiment, the mesh 520 is disposed over a second mandrel 92 having an outer diameter D₂, as shown in FIG. 9. Upon manufacture, the second tube 40 comprises an inner diameter D_{B}, as shown in FIG. 10, which is only slightly larger than the outer diameter D₂ of the second mandrel 592. Like the first tube 530, the second tube 540 may be secured in the tubular manner by melting or heat-shrinking the mesh material upon itself along the second end 524, or alternatively, by using a separate biocompatible adhesive, one or more biocompatible sutures, or other mechanisms that can maintain the structural integrity of the tubular shape for the purposes explained below.

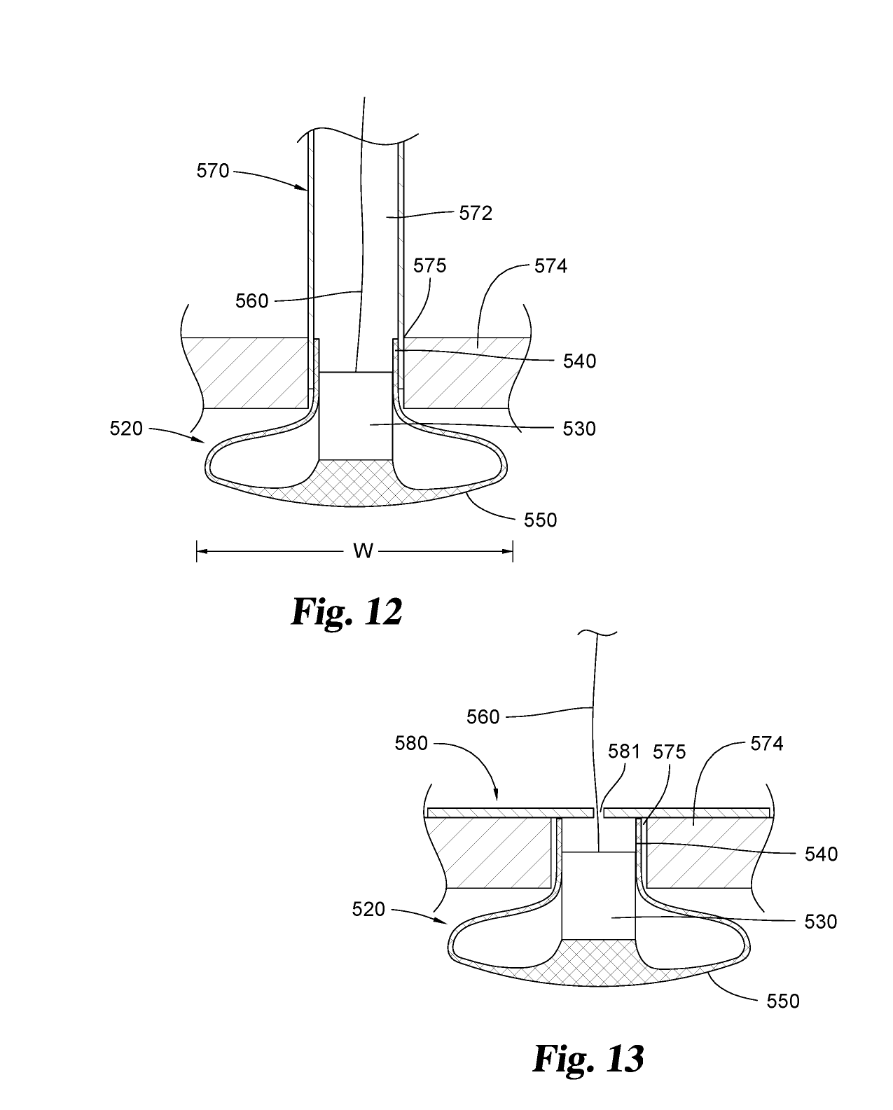

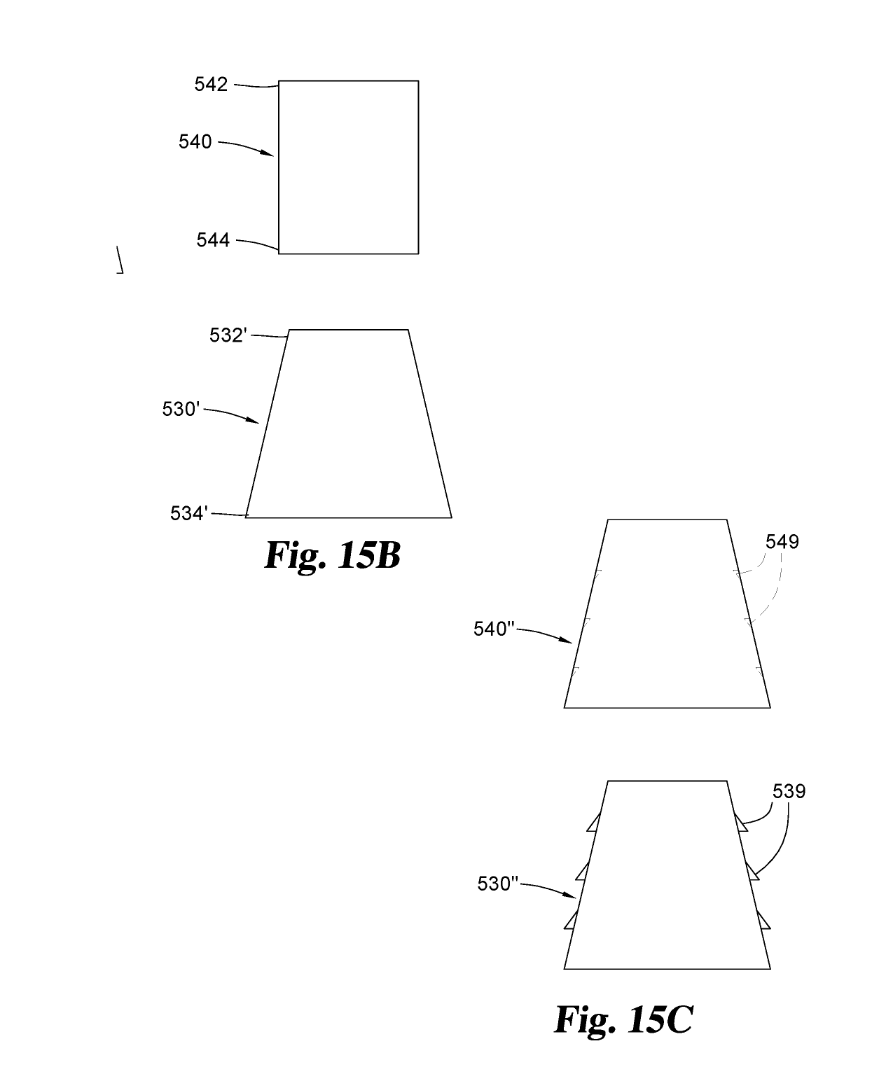

The outer diameter D_{A} of the first tube 530 is dimensioned to engage the inner diameter D_{B} of the second tube 540 using a friction fit, as explained further in FIG. 12 below. To accomplish the friction fit, the outer diameter D_{A} of the first tube 530 may be approximately equal to the inner diameter D_{B} of the second tube 40, thereby allowing the outer diameter D_{A} of the first tube 530 to snugly engage the inner diameter D_{B} of the second tube 540. In the embodiment of FIGS. 6-14, the first and second tubes 530 and 540 are depicted as being generally cylindrical with constant diameters along their lengths. In the alternative embodiments of FIGS. 15A-15C below, various alternative configurations of the first and second tubes 530 and 540 are described.

Referring still to FIGS. 9-10, the second end 524 of the mesh 520 is secured in the tubular manner such that the second tube 540 comprises a length X₂. In one embodiment, the length X₂ is less than half of the overall length of the mesh 250, where (as noted above) the overall length is measured between the most proximal and distal endpoints of the mesh 520 in a flattened state of FIGS. 6-7. Preferably, the length X₂ of the second tube 540 is between about 10.0 percent and about 38.0 percent of the overall length of the mesh 520 in the flattened state.

An intermediate portion 550 of the mesh 520, which is neither part of the first tube 30 nor the second tube 540, remains after formation of the first and second tubes 530 and 540. The intermediate portion 550 of the mesh 520 may comprise the original mesh material, e.g., untreated by heat or other techniques used to form the tubes 530 and 540, and spans from the distal end 534 of the first tube 530 to the distal end 544 of the second tube 540, as shown in FIG. 9.

The intermediate portion 550 of the mesh 520 includes the everted portion of the mesh, as shown in FIGS. 9-10, and may comprise between about 29.0 percent to about 85.0 percent of the overall length of the mesh 520, i.e., the total length of the mesh 520 minus the lengths of the first and second tubes 530 and 540. The desired length of the intermediate portion 550 of the mesh 520 may be selected based on a particular application, for example, closure of a bodily opening of a certain diameter. As will be explained further with respect to FIGS. 12-14, the intermediate portion 550 of the mesh 520 will flare radially outward to a width w to perform its intended purpose. As will be understood, the final deployed width w of the device is related to the overall length of the intermediate portion 550, i.e., if the length of the intermediate portion 550 is relatively large then the device can flare to a relatively large width w, whereas if the length of the intermediate portion 550 is relatively small then the device can flare to a relatively small width w.

Further, it is noted that an axial spacing X₃ is provided between the first and second tubes 530 and 540, as shown in FIGS. 9-10. The spacing X₃ provides a distance for retraction of the first tube 530 relative to the second tube 540, as explained further in FIG. 12 below. By varying the spacing X₃, the deployed width w of the intermediate portion 550 may be varied accordingly. For example, if a relatively large axial spacing X₃ is provided, then the first tube 530 must be retracted a relatively long distance before securely engaging the second tube 540, and during this relatively long distance the intermediate portion 550 has additional time and length to flare out to a greater width w.

Referring to FIGS. 10-11, a first tether 560 is coupled to the first tube 530, either on an inner or outer surface of the first tube 530. The first tether 560 extends proximally from the first tube 530, is disposed through the second tube 540, and extends further proximally along a length of an insertion tool 570 for actuation by a physician. A distal region of the first tether 560 may be coupled to the first tube 530 using an adhesive, mechanical member or other suitable techniques.

In a delivery state, the mesh 520 is housed within a lumen 572 of the insertion tool 570, as shown in FIG. 11. The insertion tool 570 may comprise a catheter, needle or other suitable insertion member, as noted above.

The insertion tool 570 may be advanced to a target site using various known techniques, depending on the desired treatment modality. For example, and without limitation, in one embodiment the mesh 520 may be used to treat an opening 575 of a hernia within tissue 574 of the abdominal wall, as depicted in FIG. 12. While treatment of a hernia is explained for illustrative purposes with respect to certain embodiments, it will be apparent that the systems described herein may be used in a wide range of medical procedures, as previously noted, including but not limited to repair of cardiac tissue, e.g. a right atrial appendage or any other exemplary procedures described herein.

The initial stages of the hernia repair may be performed using various techniques, with examples noted above. After gaining access to the opening 575 or target site using any of the above-referenced techniques, the insertion tool 570 may be used to deliver the mesh 520. The mesh 520 may be advanced within the lumen 572 of the insertion tool 570, e.g., using a stylet, and then may be positioned such that the second tube 540 is aligned near the distal end 573 of the insertion tool 570. At this time, a majority of the intermediate portion 550 of the mesh 520 may be disposed distally beyond the distal end 573 of the insertion tool 570. As will be appreciated, the distal end 573 of the insertion tool 570, and any of the first and second tubes 530 and 540, may comprise radiopaque markers or features that facilitate visualization of relative components positions by a physician during such delivery.

Referring to FIG. 12, in a next step, the first tether 560 is retracted proximally to cause the first tube 530 to be retracted proximally relative to the second tube 540. Optionally, a stylet may be provided within the lumen 572 of the insertion tool 570 to abut the proximal end 542 of the second tube 540 to hold it steady during retraction of the first tether 560 and coupled first tube 530. This causes the first tube 530 to engage the second tube 540, as depicted in FIG. 12.

As the first tether 560 is proximally retracted and the first tube 530 is retracted proximally relative to the second tube 540, the intermediate portion 550 of the mesh 520 expands radially outward to the width w, as depicted in FIG. 12. Locking of the first and second tubes 530 and 540 relative to one another consequently fixes the width w of the intermediate portion 550, and therefore the intermediate portion 550 is retained in its deployed state.

As explained in detail above, the first and second tubes 530 and 540 may comprise diameters that are dimensioned to securely engage each other with a friction fit, and may comprise constant diameters or tapered shapes to facilitate a secure engagement upon retraction of the first tube 530 relative to the second tube 540. A secure engagement between the first and second tubes 530 and 540 therefore may be provided.

In addition to, or in lieu of, the friction fit noted above, another locking mechanism may be used to securely hold the first and second tubes 530 and 540 relative to each other. For example, and without limitation, an exterior surface of the first tube 530 may engage an interior surface of the second tube 540 using a one-way ratcheting mechanism, which can permit incremental securement to incrementally adjust the width w of the intermediate portion 550 of the mesh 520. An example of interlocking components 539 and 549 of a ratchet arrangement is shown in the embodiment of FIG. 15C below.

If the mesh 520 is used to treat the opening 575 of a hernia within tissue 574 of the abdominal wall, the intermediate portion 550 of the mesh 520 may be anchored within the opening 575 of the hernia and/or distal to the opening 575. If deployed within the opening 575, the width w of the mesh 520 may be larger than an inner diameter of the opening 575 to secure the mesh 520 within the opening 575 using a friction fit. Alternatively, the mesh 520 may be deployed distal to the opening 575, as depicted in FIGS. 12-13, in which case the mesh 520 can assume a diameter larger than the opening 575 and provide anchoring functionality just distal to the tissue 574.

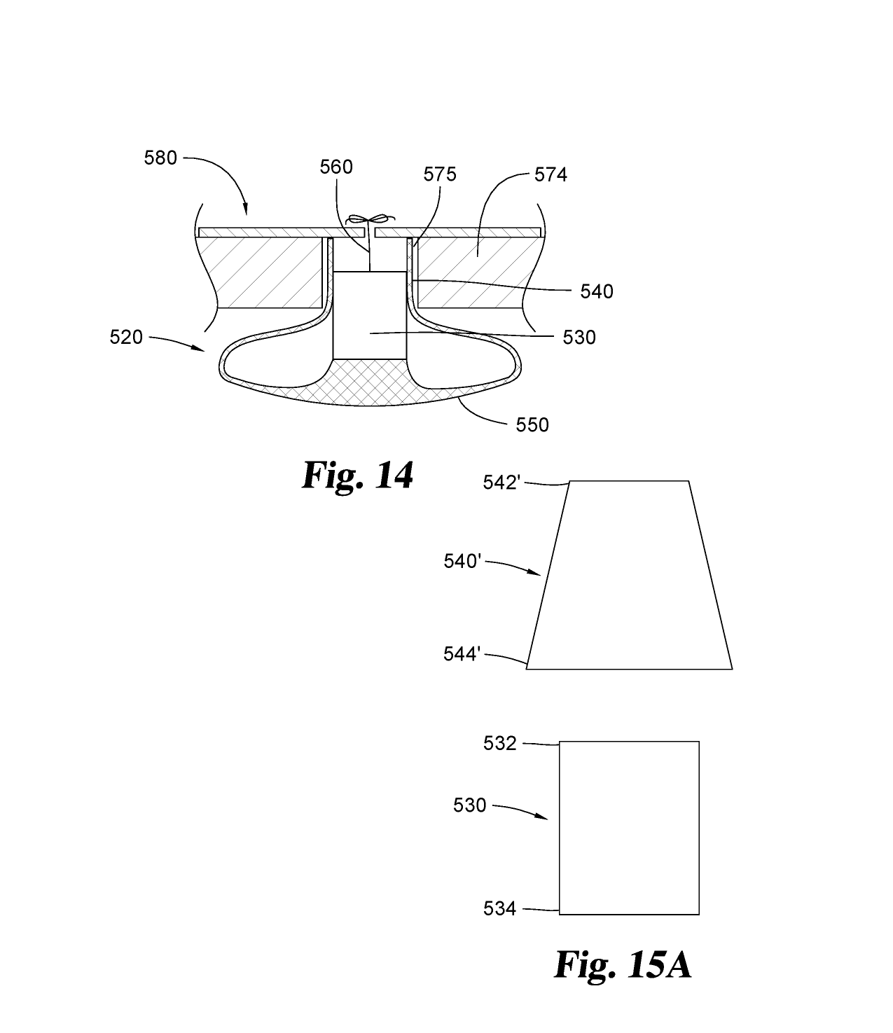

Referring to FIGS. 13-14, in a next step, a graft member 580 may be advanced distally over the first tether 560 towards the mesh 520. Properties of suitable graft members 580 are described in detail below. The graft member 580 comprises a first bore 581, which is sized to permit advancement of the graft member 580 over the first tether 560.

In use, the proximal end of the first tether 560 is disposed through the first bore 581 of the graft member 580 outside of the patient, and the graft member 580 is advanced distally relative to the first tether 560. The graft member 580 may be delivered through the insertion tool 570. Alternatively, the graft member 580 may be delivered directly through a trocar, e.g., a 5 mm trocar. When ejected from the insertion tool 570 or the trocar, the graft member 580 then is positioned in place relative to the tissue 574 using a suitable grasping device, or a pusher tube or the insertion tool 570 itself, such that the graft member 80 is adjacent to the tissue 574 and covering the opening 575, as shown in FIG. 13. In a next step, a suture tying device may be used to tie a knot for the first tether 60 to hold the graft member 580 in place.

Optionally, a second tether (not shown) may be provided in a similar manner to the first tether 560. In this embodiment, the graft member 580 may comprise a second bore, whereby the first bore 581 of the graft member 580 is advanced over the first tether 560 and the second bore of the graft member 580 is simultaneously advanced over the second tether. In this example, a suture tying device may be used to tie the first and second tethers together in a manner that secures the graft member 580 adjacent to the tissue 574 and the mesh 520. By way of example, and without limitation, one suitable suture tying device is disclosed in U.S. Pat. No. 8,740,937, the disclosure of which is hereby incorporated by reference in its entirety. Upon completion of the tying procedure, the one or more tethers may be cut by a suitable device, such as laparoscopic scissors, leaving the mesh 520 and the graft member 580 in place as shown in FIG. 14.

Advantageously, using the mesh 520, the first tether 560 (and optionally a second tether), and the graft member 580 in combination, along with the techniques described, an enhanced mesh anchoring and graft member attachment may be achieved to comprehensively treat the opening 575. Further, the coupling of the mesh 520 to the graft member 580 provides an enhanced seal relative to a plug alone, and the secure attachment of the mesh 520 to the graft member 580 may further reduce the rate of migration of the mesh 520.

The graft member 580 may comprise any suitable material for covering the opening 575 and substantially or entirely inhibiting the protrusion of abdominal matter. Particular embodiments are discussed above with respect to graft member 80.

While the exemplary embodiments herein have illustrated the use of an expandable mesh 520 for covering an opening 575 formed in the abdominal wall, the expandable mesh 520 disclosed herein may be useful in many other procedures. Solely by way of example, the expandable mesh 520 may be used to treat perforations in a visceral wall, such as the stomach wall, or could be used to treat heart defects, to prevent a duodenal sleeve from migrating, for securing a graft member to tissue for reconstructing local tissue, or various other procedures that can benefit from such an expandable mesh.

Referring to FIGS. 15A-15C, alternative embodiments are shown in which the first tube 530 and/or the second tube 540 lack constant diameters. In the embodiment of FIG. 15A, an alternative second tube 540′ comprises a tapered shape between proximal and distal ends 542′ and 544′, wherein the distal end 544′ has an inner diameter than is larger than an inner diameter of the proximal end 542′. In this embodiment of FIG. 15A, the inner diameter of the distal end 544′ of the second tube 540′ may be larger than the outer diameter D_{A} of the first tube 530 to allow the first tube 530 to be proximally retracted within the distal portion of the second tube 540, however, the inner diameter of the proximal end 542′ of the second tube 540′ may be smaller than the outer diameter D_{A} of the first tube 530 so that the first tube 530 could not be proximally retracted beyond the proximal end 542′ of the second tube 540′. In this manner, the first tube 530 may frictionally engage a region of the second tube 540′ between the proximal and distal ends 542′ and 544′.

In a further alternative embodiment of FIG. 15B, an alternative first tube 530′ may comprise a tapered shape between its proximal and distal ends 532′ and 534′. A diameter at the proximal end 532′ is smaller than a diameter at the distal end 534′ to permit retraction into the second tube 540.

In the embodiment of FIG. 15C, both first and second tubes 530″ and 540″ are tapered with proximal diameters being smaller than distal diameters. Further, in the embodiment of FIG. 15C, an exterior surface of the first tube 530″ may engage an interior surface of the second tube 540″ using a one-way ratcheting mechanism using interlocking components 539 and 549. Such a one-way ratcheting mechanism can permit incremental securement to incrementally adjust the width w of the intermediate portion 550 of the mesh 520. In addition to the friction fit and one-way ratcheting mechanism options, it is contemplated that other coupling methods may be used to secure the first and second tubes together, including but not limited to magnetic couplings, knobs or beads that interlock in notches, or other mechanical arrangements.

Examples of closure elements are disclosed below, which may use elements or features described above. For example, mesh 520 with ends 530 as described above may be used as (or as part of) the closure elements 720, 722 discussed below.

Referring generally to the drawings, there are shown embodiments of parts of a system 720 for closing a hole in tissue, for example cardiac tissue. Such a system may include one or both of a closure device 722 and a placement device 724. As will be discussed further below, closure device 722 is initially placed within placement device 724. When placement device 724 is adjacent or through a tissue opening, closure device 722 is moved out of placement device 724 to cover the tissue opening, and is fixed in place to permit or promote healing.

Closure device 722 in the illustrated embodiment is a two-piece device, having a first or distal closure element 730 and a second or proximal closure element 732, which can be similar or identical to, in whole or in part, anchor 20 and/or mesh 520 described above. “Distal” and “proximal” in this context are defined as above, referring to relative position with respect to the direction of travel of closure device 722 and/or placement device 724, “distal” being generally toward or beyond a tissue hole or opening to be closed, and “proximal” being generally toward the operator along that direction of travel. Closure element 730 is intended to engage tissue and cover an opening through it on the distal side of the tissue, i.e. the side beyond a hole through the tissue. Closure element 732 is intended to engage tissue and cover an opening through it on the proximal side of the tissue, i.e. the side approached first by placement device 724. Closure elements 730 and 732 are linked together prior to insertion into a patient's body or on or into a delivery device in particular embodiments, as discussed further below.

Closure element 730 in the illustrated embodiment is made of a mesh 734, and in particular embodiments are bioresorbable, non-bioresorbable, and/or of a biologic material. Such materials may be or include those described above with respect to anchor 20 and/or mesh 520, e.g., polypropylene, polyethylene, glycolide/L-lactide copolymer, PTFE, nylon, polyurethane, PEEK, PLGA, PGA, polycaprolactone, carbothane, polydioxanone, or copolymers of such constituents. Mesh 734 as illustrated includes a number of interstices 736 among a solid but flexible material that are or have the appearance of crossed strand(s) or similar linear member(s) 737.

Closure element 730 has first and second ends 738, 740 in the illustrated embodiment which are narrowed or closed. In one example, a sheet or length of mesh 734 is rolled or otherwise formed around an axis into a cylinder or other longitudinally closed shape having opposing open ends 738 and 740. In such embodiments, mesh 734 has a central volume 742 between ends 738 and 740. End 738 is narrowed or closed to form a tube (e.g. with a passage), a closed mass, or other tip. For example, narrowing or closing can be accomplished by heat-shrinking a portion of end 738 to form a tube with a passage having a diameter substantially smaller than a nominal diameter of central volume 742, e.g. one-third to one-tenth of such nominal diameter or smaller. As another example, narrowing or closing can be accomplished by chemically or thermally fusing end 38 to form a closed mass as a tip. Techniques such as those described above and/or in U.S. patent application Ser. No. 14/748,992 may be used. End 40 is similarly narrowed or closed, preferably to form a tube with a small passage through it. Closure element 30 thus has an intermediate portion 44 of mesh 34 longitudinally in between narrowed or closed ends 38 and 40, with volume 42 being within intermediate portion 44 and bounded by mesh 34.

In particular embodiments, end 738 (and potentially other end(s) of closure elements 730, 732) is or includes a radiopaque marker. For example, such a marker may be a tube of biocompatible metal (e.g. gold, platinum, tungsten-, zinc-, iron-, and/or magnesium-based metals) or appropriate bioresorbable materials. Exemplary markers have open ends and an interior and exterior, and in some embodiments include a side opening through a side wall. It is encased by mesh 734 of the particular closure element. Such integration is possible where the mesh 734 is able to exist as a fluid mass and can undergo a phase change to a solid mass. With end portion of mesh 734 treated to become a fluid mass (as by heating, chemical curing, or applying electric or magnetic fields), pressure is applied to direct the fluid mass into and around the tubular marker, e.g. in or through the open ends and/or the side opening. The fluid mass then undergoes a phase change to solid (as by cooling) so that the solid mesh material encases the tubular marker. The interior of the marker may be occluded by the mass entirely, or a lumen can be left through the encased marker. In this way, the marker is securely anchored with respect to the mesh 34 while covering any rough edges on the marker. Such markers serve to indicate when the ends of one or both of closure elements 730, 732 are pulled together, e.g. to indicate the shape of the compressed or collapsed mesh and indicate if the mesh compression is distorted.

Closure element 730 in the illustrated embodiment is double-inverted, meaning that each end 738, 740 is inverted so that the narrowed or closed portions point into or are within volume 742. For example, closure member 730 may be made by forming a sheet of mesh 734 into a tube with open ends (which will become ends 738 and 740). In that tube form, there is an exterior surface 746 with edges 747 surrounding an inner space, which will form the volume 742 of closure member 730. An interior surface 48 faces that volume 742. Forming an inverted end includes turning the edge 747 into the inner space, so that the edge is inside of a portion of the interior surface 748. The end is narrowed or closed, as noted above (e.g. by heat-shrinking, chemical treatment), so that the edge remains inside volume 742 of closure 730. In such an example, a portion of the exterior 746 of the mesh 734 folds over itself, and a curved or folded part of that exterior 746 forms an exterior end 750 of closure 730, with end 738 inverted into volume 742. It will be understood that the narrowing or closing of the end(s) can occur prior to or after inversion. As noted, closure element 730 is double-inverted, so that end 40 is also inverted identically or similar to end 738.

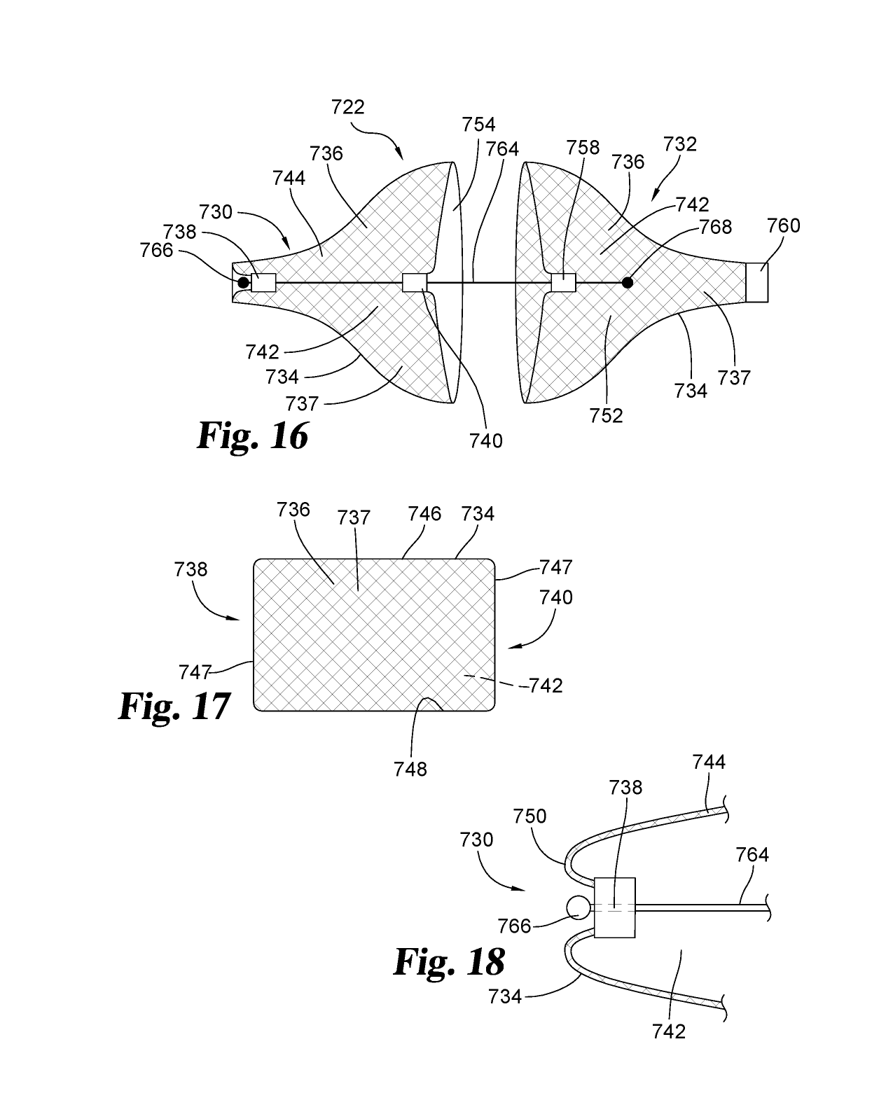

In particular embodiments, some or all of closure element 730 includes a sheet or mass of therapeutic or healing material 754 (e.g. graft material 80, 580 discussed above) which may at least partially block fluid flow and/or assist in tissue growth and contribute or assist the healing process. As an example, a sheet, layer or other portion of SIS (small intestinal submucosa) may be placed to line the inside of mesh 734 (e.g. within volume 742) or fixed to an outside portion of mesh 734. A layer 754 is indicated in FIG. 16 fixed to the outside of mesh 734 to cover most or all of the surface around end 740, or at least so that when closure element 730 is flattened as discussed further below, the sheet 754 covers at least part of a tissue opening to be repaired or healed.

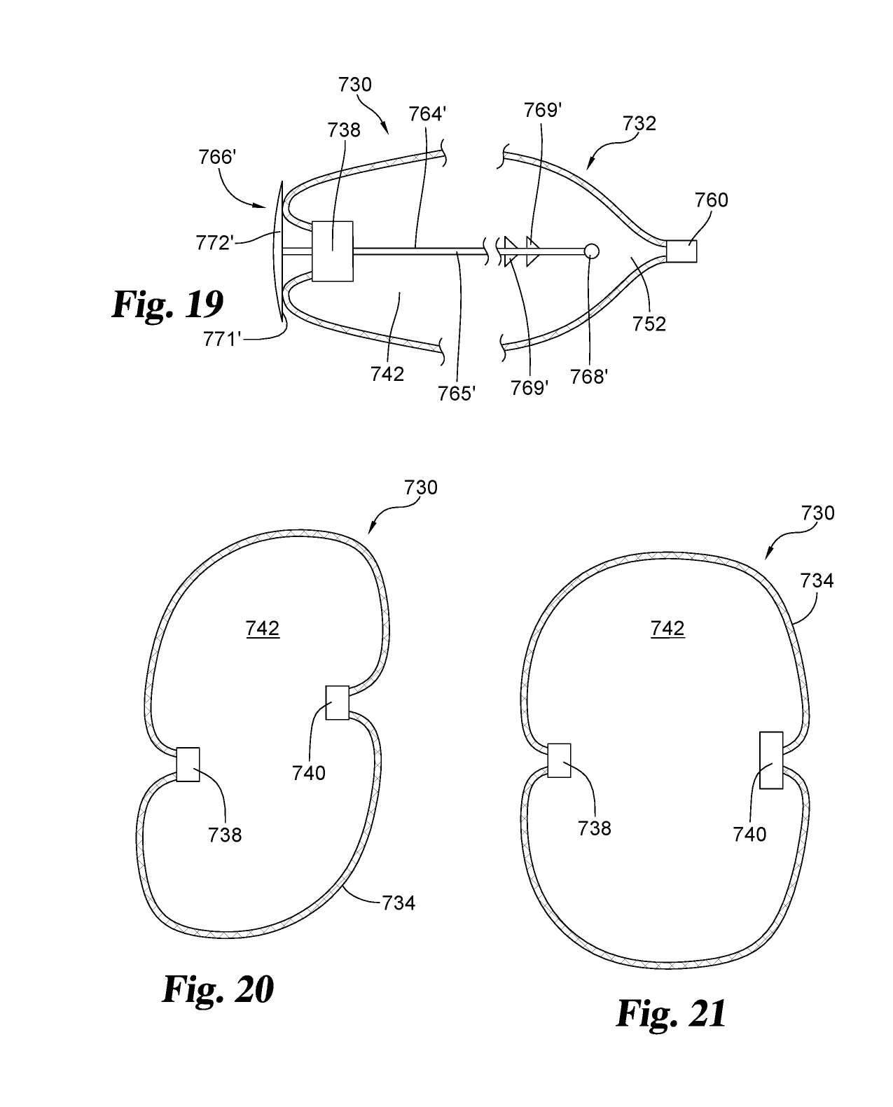

Closure element 732 is for proximal placement, i.e. on the side of the tissue that is first reached or approached by placement device 724, and is similar to closure element 730 in particular embodiments. For example, closure element may be identical or essentially identical to closure element 730 as described above, having mesh 734 as a body enclosing a central volume 752, with first and second ends 758, 760 in the illustrated embodiment which are narrowed or closed. In a particular embodiment, closure element 732 is similar to closure element 730 as described above, but has one inverted end 758 and one non-inverted end 760 (see FIGS. 16, 19). End 758 is inverted and closed or narrowed as described above. End 760 is closed or narrowed as described above, but an exterior surface of mesh 734 of closure element 732 is not folded in on itself, and so end 760 does not enter or point into volume 752. Rather, in this embodiment end 760 points generally away from volume 752. In a particular embodiment, ends 758 and 760 are aligned along a common longitudinal axis that extends through volume 752. It will be understood that in other embodiments closure element 732 is double-inverted, like closure element 730, rather than single-inverted.

Closure elements 730 and 732 are joined by a filament or tether 764 in specific embodiments. Tether 764 has two ends 766, 768 that are enlarged, as with knots or beads, that are at least slightly larger than any opening through ends 738, 740, 758, 760 of closure elements 730, 732. Additional beads, knots or other enlarged portions may be present between ends 766 and 768 for adjustability in locking closure elements 730, 732. Tether 764 extends through end 738 of closure element 730, with end 766 of tether 764 outside of volume 742 and within, engaged with or beyond end 738 of element 730, so as to be fixed or otherwise connected to end 738. From end 738, tether 764 passes through both ends 738 and 740 and volume 742 of element 730. From closure element 730, tether 764 extends across any gap that may exist between closure elements 730 and 732, and then passes through end 758 and into volume 752 of closure element 732. It will be understood that in embodiments in which tether 764 is fixed to end 738 of closure element 732, other ways of fixation such as adhesives or fusion, could be used to fix tether 764 to end 738 or another part of closure element 730. In the illustrated embodiment, tether 764 is connected to end 738 at a point within the inner space created by the fold of the inversion of end 738. Tether 764 is not fixed with respect to end 740 of closure element 730, but can be moved through end 740, as by pulling. Likewise, tether 764 is not fixed to end 758 of closure member 732, but can be moved through ends 758 and end 768 of tether 764 can be forced through end 760 of closure element 732, as by pulling. Pulling on tether 764 can collapse closure member 730 toward closure member 732 and toward tissue between members 730 and 732.

As will be discussed further below, a tensioning or control line 770 is looped through end 768 of tether 764, passing into and out of closure element 732 via the opening through end 750 of closure element 732. In particular embodiments, line 770 passes through a bight, hole or knot in tether 764. When line 770 is pulled or otherwise placed in tension, it places tension on tether 764 and thereby pulls on end 738 of closure member 730, pulling or flattening closure member 730 toward tissue and closure member 732. Control line 770 is also a part of the procedure to flatten closure element 732 with respect to tissue, as will be discussed further below.

In an alternative embodiment, a filament in the form of a solid stem 764′ is provided in place of tether 764. Stem 764′ in the illustrated embodiment has an elongated body 765′ with a flat end 766′ and an opposite end 768′. In the illustrated embodiment body 765′ and ends 766′ and 768′ are monolithic, e.g. formed or created as a single piece of the same material. Such materials are preferably a bio-resorbable material that has sufficient strength to hold the two closure elements 730, 732 together for a time sufficient to allow the closure elements 730, 732 to be encapsulated and sealed by bodily tissue. Body 765′ includes one or more protrusions 769′ to act as stop points or barbs between end 768′ and flat end 766′. In particular embodiments, protrusions 769′ have surfaces facing flat end 766′ that are perpendicular to or form an acute angle with a longitudinal axis of body 765′, to form stop surfaces as will be discussed further below. End 768′ is adapted to be engaged to a suture or control line 770 (as discussed further below), for example having a loop, bight or eye through which control line 770 can extend and/or be attached to body 765′. Flat end 766′ in a particular embodiment is substantially planar along a surface 771′ that adjoins body 765′, and convexly curved along a surface 772′ opposite surface 771′.

In this embodiment, stem filament 764′ is fixed to or otherwise engaged with closure member 730. For example, body 765′ is inserted through the closed or narrowed end 738 of closure member 730, with flat end 766′ abutting a portion of closure element 730 that is distal of the inverted end 738 so as to anchor stem 764 to the closure element 730. Body 765′ extends through closure element 730 and into or through closure element 732 in an initial condition, and is adapted to extend through and away from end 750 of closure element 732 when placed in the body. In particular embodiments, body 765′ can extend through each of ends 738, 740, 758, 760 of the closure elements 730, 732, and in other embodiments body 765′ need not pass through one or more of those ends, but can pass through the mesh of one or more parts of closure elements 730, 732.

When closure device 722 is initially prepared, end 768 of tether 764 or protrusions 769′ of body 762′ extend at least through end 738 and into volume 742 of closure element 730. Control line 770 extends from tether 764 or body 765′, and depending on how far tether 764 or body 765′ is initially placed through closure element 730 and/or 732, line 770 extends through closure element 730 and 732, exiting closure element 732 via narrowed or closed end 760. As closure elements 730, 732 are being placed, they are compressed so that tether 764 or stem 764′ holds one or both of them in a compressed state. For example, once closure element 730 is placed (as discussed further below), line 770 may be pulled, so that tether 764 or stem 764′ is pulled, and end 766 of tether 764 or one or more protrusions 769′ of body 765′ are forced through end 760 or another portion of closure element 732. End 766 of tether 764 or flat end 766′ of stem 764′ pulls the distal portion (or end 738) of closure element 730 toward the proximal portion (or end 740) of closure element 730, compressing closure element 730. One or more protrusions 769′ can engage a proximal portion (or end 740) of closure element 730 to prevent re-expansion of closure member 730. Similarly, further pulling of line 770 can draw body 765′ through a distal portion (or end 758) of closure element 732 and/or through a proximal portion (or end 760) of closure element 732 to compress closure element 732 on itself and/or toward closure element 730, to finally fix closure device 722 against tissue. Thus, tether 764 or stem 764′ passes between and within closure elements 730, 732, with end 766 of tether 764 or flat end 766′ of stem 764′ on the distal outside of closure element 730, and at least one protrusion 769′ of body 765′ on the proximal outside of closure member 732.

The inventors have further found that there is an advantage in some uses of closure device 722 of reducing the compressed, in-use height of one or both of closure elements 730, 732. By “height” in this context is meant the dimension measured outward from the tissue to which the closure elements are applied. To address those cases where space is minimal or where a smaller closure is otherwise indicated, closure element 730 is prepared so that ends 738 and 740 are offset from each other within volume 742. As seen in FIG. 20, ends 738 and 740 are inverted and narrowed or closed as described above but are arranged non-symmetrically, so that each end 738, 740 is to one side of the other. This allows closure element 730 to be compressed so that ends 738 and 740 move past each other, with less or no contact or other interference with each other as compared to a configuration as described above in which ends 738, 740 are aligned or coaxial. In this embodiment, tether 764 or stem 764′ is fixed to end 738, as discussed above, but does not extend through end 740. Rather, tether 764 or stem 764′ passes through mesh 734 alongside end 740, and on to closure element 732, as discussed above. It will be understood that a similar configuration could also or instead be applied to closure element 732.

In another embodiment (FIG. 21), closure element 730 has ends 738 and 740 that are aligned, but with one of the ends 738 or 740 of a larger diameter than the other. For example, end 740 is inverted and narrowed as discussed above, to a given diameter. End 738 is inverted and narrowed as discussed above to a diameter smaller than that of narrowed end 740. Tether 764 or stem 764′ extends through both ends 738 and 740. When closure element 730 is compressed, end 738 is pulled toward end 740 so that end 738 enters at least partially into end 740. It will be understood that in other embodiments end 740 may be smaller than end 738, and that similar configuration(s) could also or instead be applied to closure element 732.

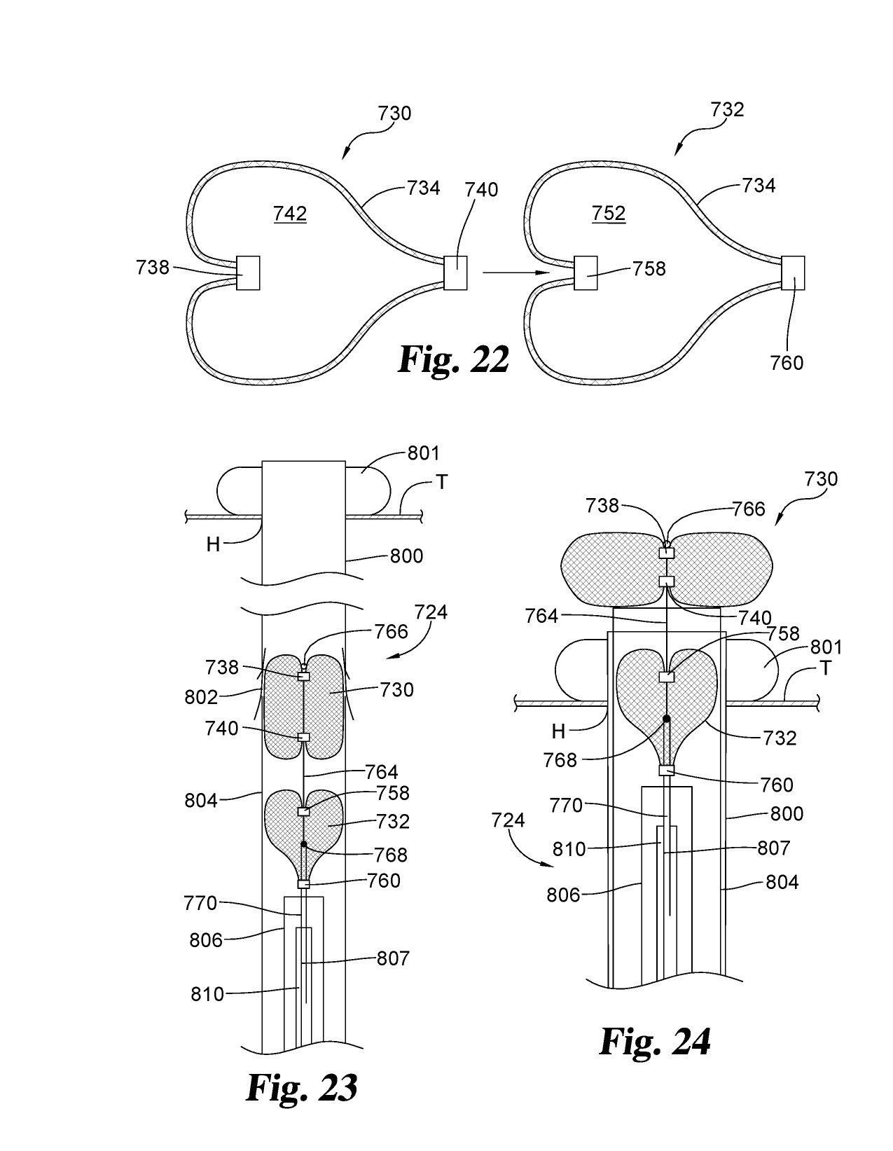

In another embodiment (FIG. 22), closure element 730 is not a double-inverted member, but is instead a single-inverted member like the illustrated embodiment of closure element 732. Thus, in this embodiment end 738 is inverted as discussed above, but end 740 is not inverted, like end 760 of closure element 732. One or both of ends 758, 760 of closure element 732 have a diameter greater than that of end 740 of closure member 730, as in the above discussion. Tether 764 or stem 764′ passes through each of ends 740 and 758 initially, and in use through end 760, in this embodiment. When closure elements 730, 732 are compressed, end 740 of closure element 732 passes through the tissue hole to be closed or sealed, and may enter one or both of ends 758, 760 of closure element 732.

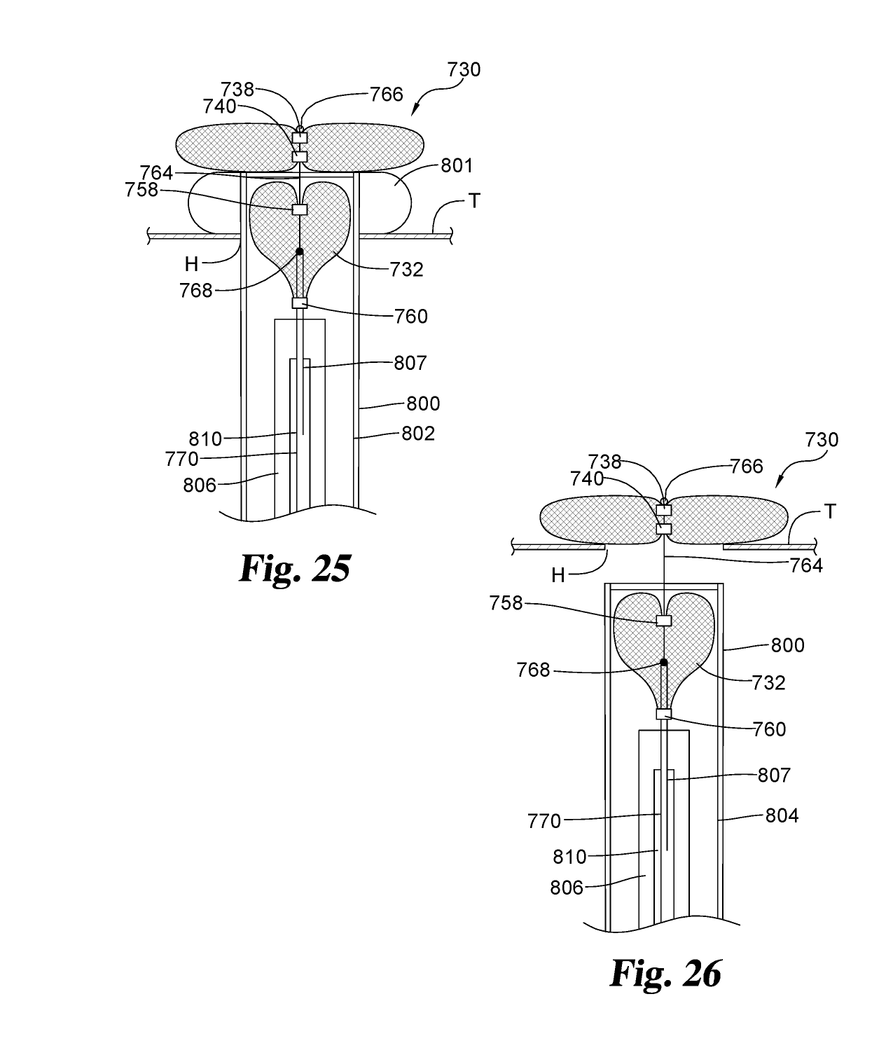

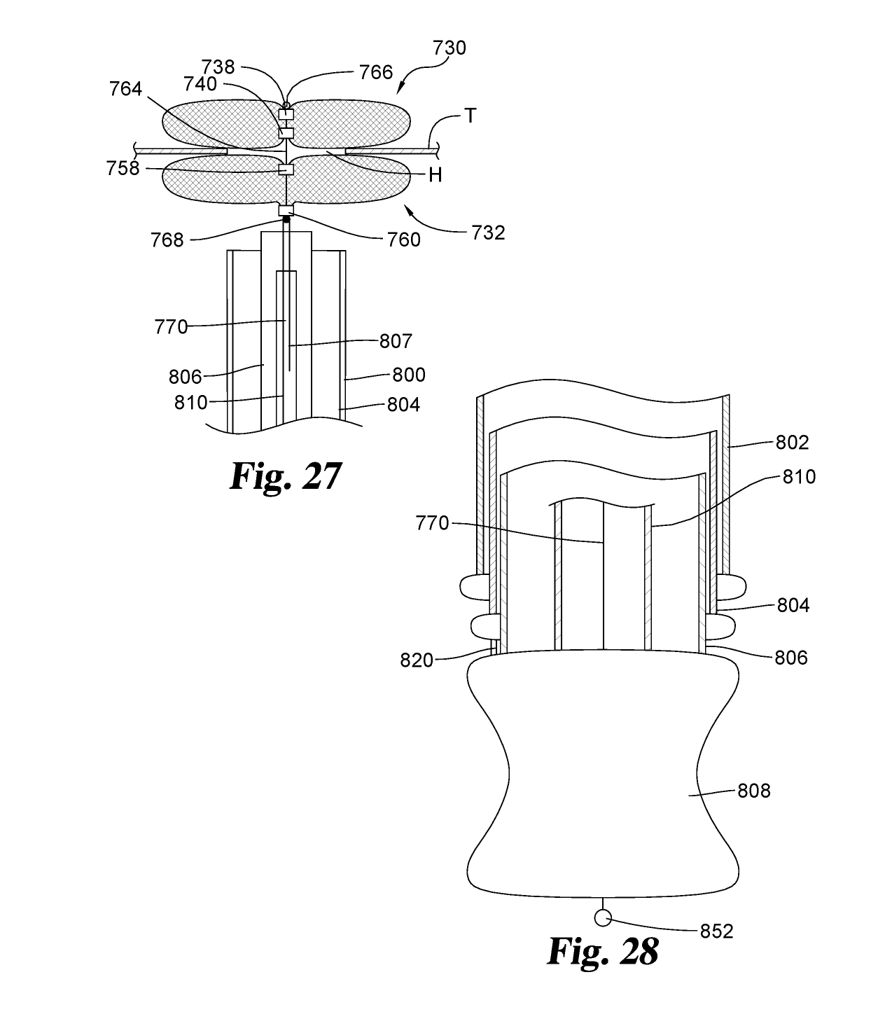

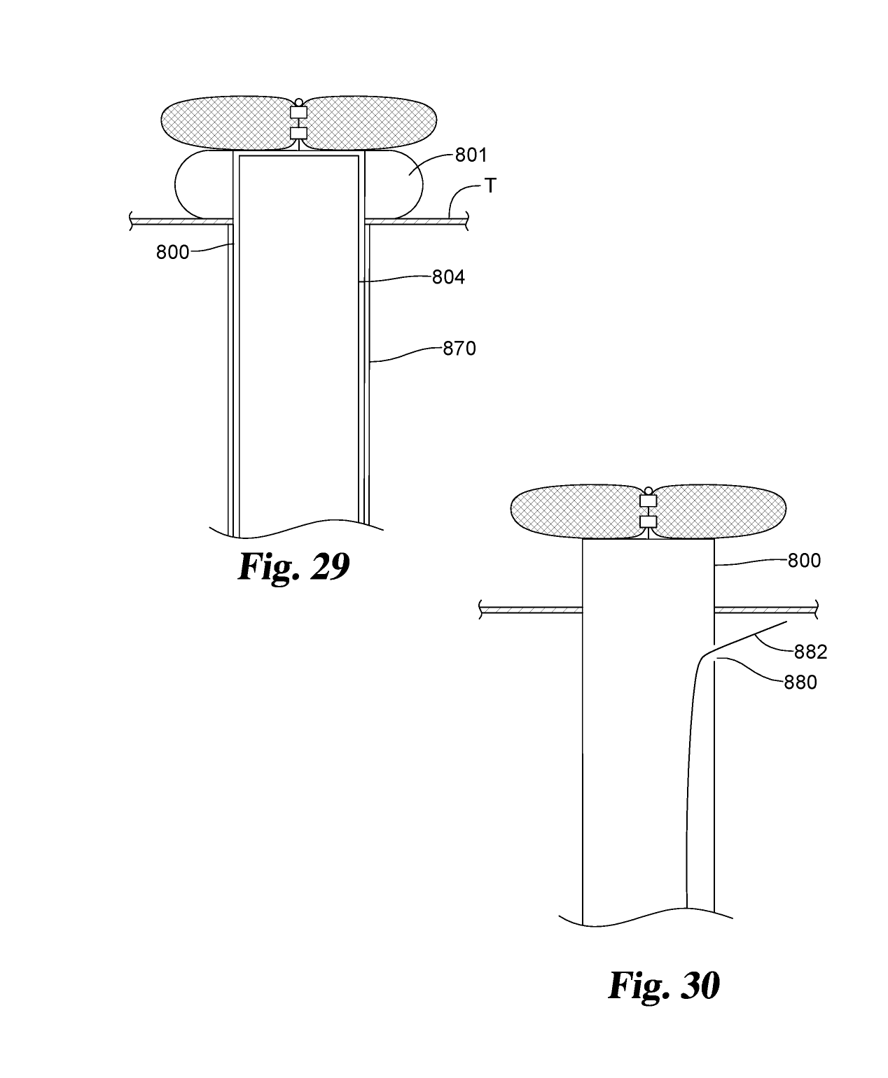

Closure device 722 may be placed in the body in a minimally-invasive manner, e.g. by obtaining percutaneous access to a blood vessel, organ or other part of the body and moving closure device 722 with or through a catheter or other tube to the desired location. It will be understood that closure device 722 may be placed at the desired location in the body via open surgery or other procedures as well. An embodiment for placement device 724 for minimally-invasive placement of closure device 722 is shown schematically in FIGS. 23-28. This embodiment of placement device 724 is intended for insertion through a previously placed sheath 800 that allows access to or is placed through a hole H in tissue T to be closed. In this example, sheath 800 extends from within a right atrial appendage (RAA) through hole H in tissue T to the pericardial space, in which balloon 801 of sheath 800 is inflated to anchor sheath 800.

Device 724 includes three tubular members 802, 804 and 806 to which a handle 808 is connected, in the illustrated embodiment. As will be discussed further below, device 724 is inserted through delivery sheath 800, which in one example is a 14 French tubular sheath that has been advanced through and anchored with respect to hole H. In that example, tubular member 802 is a 14 French peel-away tube around and at the distal end of member 804, which can be a 12 French delivery sheath. Member 806 is a pusher and/or guide cannula, which is slidable within member 804. Thus, member 804 is initially at least partially inside peel-away member 802, and member 806 is within member 804. In particular embodiments, a further tubular member 810, extending through member 806, is provided as a guide cannula for control line 770. Control line 770 extends through member 810, and in a particular embodiment line 770 is a thin braided stainless steel cable. Line 770 has a proximal end connected to handle 808, and extends out of the open distal end of member 810, through end 760 of closure element 732, looping through tether 764 or stem 764′ (as discussed above), and returning through end 760 and into member 810. In this way, line 770 is doubled initially within member 810, so that a free end 811 is in member 810 and generally points proximally. Member 804 can fit into sheath 800 while containing closure device 722. In an initial (pre-usage) configuration, closure element 730 is within member 802, and closure element 732 is within member 804, which is immediately or closely adjacent closure element 730. Pusher member 806 is initially proximal or rearward of closure element 732. As device 724 is inserted into the delivery sheath 800, peel-away member 1802 is pulled apart and removed, leaving closure member 730 within sheath 800, and delivery member 804 (with closure element 732 inside) behind closure element 730 and also within sheath 800.

Embodiments of system 720 (e.g. placement device 724 and/or one or both of closure elements 730, 732) can be configured to accommodate passage of a wire guide. For example, such a wire guide may run through each closure element 730, 732 and through placement device 724 and into sheath 800 that is in hole H. The wire guide may run alongside or within the pusher member 806, or may run through a separate lumen through or alongside device 724. If the wire guide passes through closure elements 730, 732, it may do so through mesh 734 at location(s) other than at one or more of ends 738, 740, 758, 760. Such a wire guide serves both to help align the closure elements 730, 732, and to provide a navigation pathway that may be left behind in the event that use of system 720 needs to be abandoned and the closure process re-started. In such a case, the wire guide provides a guide path for subsequent closure devices to approach and close hole H.

As noted, the illustrated embodiment of sheath 800 includes a balloon 801 at or near a distal end, to anchor delivery sheath 800. When sheath 800 extends through a hole, to deliver therapeutic devices or compositions or for other purposes, balloon 801 is inflated on the distal side of the hole in order to anchor delivery sheath 800 in place. With sheath 800 so anchored, and all desired procedures via sheath 800 having been performed, the user inserts placement device 724 in its initial configuration into sheath 800 and peels away member 802, as indicated above. Such insertion and advancing into sheath 800 may be accomplished over a wire guide that passes through or along device 724. Device 724 without member 802 (i.e. members 804 and 806, connected to handle 808 and including the features noted above within members 804 and/or 806) is then pushed through sheath 800 so that closure element 730 emerges from the distal end of sheath 800. In particular embodiments, moving delivery member 804 forward from or using handle 808 pushes closure element 730, so that closure element 730 is pushed outside of sheath 800. Member 804 and/or handle 808 connected to it is preferably locked to sheath 800 outside the patient's body, and deployment of closure element 730 can be visualized (e.g. by fluoroscopy).

When closure element 730 has emerged from member 804, distal end 738 is generally away from tissue T through which hole H extends. End 740 is directed toward tissue T, so that exterior end 750 (with folded-over mesh and in some embodiments healing material, as noted above) faces hole H. Tension is maintained on tether 764, by pulling back at least slightly on member 810 (which may be connected to or locked with members 804 and/or 806). Such pulling provides tension via control line 770 to tether 764 and on to end 738 of closure element 730, flattening closure element 730 against the distal end of sheath 800.

At this point, balloon 801 is deflated to permit withdrawal of sheath 800 from hole H. Sheath 800 and device 724 are withdrawn together until closure element 730 engages the distal surface of tissue T (e.g. the pericardial side of the RAA wall). Again, the user can visualize the site to confirm that closure element 730 is against the tissue and/or to confirm that sheath 800 is out of hole H (e.g. fully on the cardiac side of the RAA wall). The position of member 806 (e.g. with member 810) and line 770 is then maintained as member 804 and sheath 800 are withdrawn further to expose and deploy the middle of tether 764. The user can confirm (e.g. by visualization under fluoroscopy) the spacing between closure element 730 and the distal end of member 804 and/or that the distal end of member 804 is clear of the RAA wall. Again maintaining the position of member 806 (e.g. with member 810) and line 770, member 804 and sheath 800 are further withdrawn to expose and deploy closure element 732 from within member 804. Visualization of that deployment can be performed.

With closure element 732 out of member 804 and its distal end 758 generally facing tissue T, the operator holds the position of line 770 while advancing pusher member 806 against proximal end 760 of closure element 732. Member 806 pushes end 760 over enlarged end 768 of tether 764, forcing end 768 through end 760 and flattening closure element 732. As noted above, enlarged end 768 of tether 764 is larger than an opening through end 760 of closure element 732, so that once tether end 768 is forced through end 760, closure device 722 is locked. That is, closure elements 730 and 732 are flattened against their respective sides of tissue T, and tether 764 locks them together, preventing ends of the closure elements from passing over enlarged ends 766, 768 of tether 764.

With closure device 722 locked, line 770 is maintained in position while member 810 is withdrawn sufficiently to allow the free end 811 of line 770 to escape the distal end of member 810. Line 770, as noted above, is looped so as to have free end 811 within member 810, and in particular embodiments, free end 811 bends outward when free of member 810. With free end 811 outside of member 810, line 770 is withdrawn (e.g. via handle 808). Free end 811 is pulled through end 768 of tether 764 and from closure element 732, and away from the treatment site. The remainder of device 724 (including members 804 and 806) and sheath 800 can then be withdrawn, over a wire guide if present. Closure device 722 remains in the above-noted locked condition to allow healing of hole H.

An exemplary embodiment of the operational (distal) end of device 724 is shown schematically in FIG. 28. Peel-away sheath 802 is shown as outermost, with distal closure element 730 within it. An inner catheter or tube 804 is within peel-away sheath 802, and its distal end is closely adjacent to closure element 730. Proximal closure element 732 is within inner catheter or tube 804. A pusher or guide tube or cannula 806 is within inner catheter or tube 804, and in particular embodiments a cannula 810 for control line 770 is provided. Control line 770 extends through member 806 and/or member 810, is threaded through end 768 of tether 764, and returns to member 806. Proximally, each of members 804, 806 (and member 810 if present) and line 770 are connected to handle 808.

The representation of handle 808 shows pusher catheter 806 connected directly to it, with peel-away sheath 802 and inner catheter or tube 804 around pusher catheter 806. A lock 820 may connect handle 808 and tube 804 in particular embodiments. The connection of handle 808 and line 770 is not shown in that figure. In the illustrated embodiment, handle 808 includes a body 850 shaped and configured for holding and maneuvering by hand. Three actuators are placed on or in handle 808. A control line actuator 852 may include a pull ring and a shaft connected to line 770. Actuator 852 maintains line 770 in position, and by pulling actuator 852, line 770 is pulled or placed in tension. Additional actuators may be connected to members 804 and/or 806 to permit relative motion of them with respect to each other or other parts of device 724. It will be understood that one or more actuators can be assembled in series to automate several actions with essentially one motion.

One problem that has occurred with placement of delivery sheaths or similar devices through tissue openings is that when the sheath is withdrawn, the friction of the withdrawing sheath can stretch or move the tissue plane through which the opening extends. For example, in an procedure in which a hole is created in the thin wall of the right atrial appendage for a delivery sheath, when the sheath is withdrawn through the hole the appendage wall will tend to invaginate into the right atrium, which is undesirable. To address that problem, an outer sheath 870 may be placed over the delivery sheath (800 in the illustrated embodiment) that is anchored by a balloon 801. While balloon 801 is inflated, sheath 870 is moved to a position so that its distal end is just proximal to the hole (e.g. engaging or closely adjacent to tissue around the hole). When the delivery sheath 800 is withdrawn (following deflation of balloon 801), the distal end of sheath 870 supports the wall of the tissue plane through which sheath 800 is withdrawn.

Alternatively, delivery sheath 800 may include a lumen (either the same or a different lumen from that containing device 724) and a communicating side port 880 located a sufficient distance below balloon 801 to be at least partially below an inner tissue wall surface when balloon 801 is inflated and anchoring on the outer tissue wall surface. A wire 882 fabricated from a shape-memory material (e.g. Nitinol) is within the lumen in an unexpanded or restrained shape or configuration. When withdrawal of sheath 800 is desired, wire 882 is advanced through the lumen and at least partially out of port 880. As wire 882 emerges from port 880, it assumes an expanded shape, e.g. an expanding helical shape. Wire 882 presses against the wall of the tissue plane as it is advanced, providing counter support for the tissue as the sheath 800 is withdrawn.

It will be understood that port 880 may also be used for other purposes, in addition to or instead of placement of wire 882. For example, once sheath 800 is in place or close to it, a contrast medium can be injected through the lumen and the side port 880 to help visualize the hole or tissue surrounding it. Similarly, side port 880 may be used as a flush port.

The above discussion of closure of a hole in an organ or other tissue is generally applicable to a number of types of openings, whether occurring naturally (e.g. a fistula) or artificially (e.g. through trauma or for passage of a therapeutic or diagnostic device). In particular embodiments, as noted above, the devices and methods described herein can be used for repairing a hole through a right atrial appendage opened for passage of treatment devices to the heart. In such an embodiment, the tissue T is part of the right atrial appendage, separating the appendage's interior from the pericardial space.

While the subject matter herein has been illustrated and described in detail in the exemplary drawings and foregoing description, the same is to be considered as illustrative and not restrictive in character, it being understood that only the preferred embodiment(s) have been shown and described and that all changes and modifications that come within the spirit of the disclosure are desired to be protected. It will be understood that structures, methods or other features described particularly with one embodiment can be similarly used or incorporated in or with respect to other embodiments.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

The following numbered clauses set out specific embodiments that may be useful in understanding the present invention:

1. A closure for an opening in tissue, comprising:

a first closure element, the first closure element having a first mesh enclosure, the first mesh enclosure having a first distal narrowed end and a second proximal narrowed end and a central volume, each of the first and second ends being inverted so as to be within the central volume of the first mesh enclosure, each of the first and second ends being surrounded by respective external surfaces of the first mesh enclosure;

a second closure element, the second closure element having a second mesh enclosure physically separate from the first mesh enclosure, the second mesh enclosure having a third distal narrowed end and a fourth proximal narrowed end and a central volume, the third end being inverted so as to be within the central volume of the second mesh enclosure, each of the third and fourth ends being surrounded by respective external surfaces of the second mesh enclosure;

a tether joining the first and second closure elements in an initial configuration prior to delivery of the closure elements to the opening, the tether having first and second enlarged ends, wherein the tether extends through at least the first narrowed end, parallel to and alongside the second narrowed end and into the second closure element, so that the first enlarged end of the tether is outside the first closure element adjacent or engaging the first end and the second enlarged end of the tether is within the central volume of the second closure element and positioned to pass through the fourth narrowed end,

wherein the first closure element is adapted to engage a distal side of the tissue having the opening, and the second closure element is adapted to engage a proximal side of the tissue, and the tether is adapted to pass through the opening.

2. The closure of clause 1, further comprising a sheet of bioresorbable material fixed to the first closure element adjacent or over the second narrowed end.
3. The closure of any of clauses 1-2, wherein the sheet is fixed to an external portion of the first mesh enclosure.
4. The closure of any of clauses 1-2, wherein the sheet is fixed to an internal portion of the first mesh enclosure.
5. The closure of any of clauses 1-4, further comprising a sheet of bioresorbable material fixed to the second closure element adjacent or over the third narrowed end.
6. The closure of any of clauses 1-5, wherein each of the enlarged ends of the tether comprise a bead or a knot.
7. The closure of any of clauses 1-6, further comprising a control line looped through the second enlarged end of the tether.
8. The closure of clause 7, wherein the control line passes through the fourth narrowed end.
9. The closure of any of clauses 1-8, fitted within a delivery device.
10. The closure of any of clauses 1-9, wherein the first and second narrowed ends are aligned with each other.
11. The closure of any of clauses 1-10, wherein the third and fourth narrowed ends are aligned with each other.
12. The closure of any of clauses 1-11, wherein the first and second narrowed ends are laterally offset with respect to each other.
13. The closure of any of clauses 1-12, wherein one of the first and second narrowed ends is larger in diameter than the other of the first and second narrowed ends.
14. The closure of any of clauses 1-13, wherein at least one of the first and second closure elements are adapted to expand in width during placement as at least one of the enlarged ends of the tether and at least one of the narrowed ends of at least one of the closure elements move with respect to each other.
15. A device for closing an opening in tissue, comprising:

a delivery device having a first peel-away catheter, a second tube within the first peel-away catheter, and a pusher tube within the second tube; and

the closure device of claim 1 fitted within the delivery device.

16. The device of clause 15, wherein the first closure element is within the first peel-away catheter and the second closure element is within the second tube.
17. The device of any of clauses 15-16, further comprising a control line looped through the second enlarged end of the tether.
18. The device of clause 17, further comprising a control cannula extending through the pusher tube, and wherein the control line extends through the control cannula.
19. The device of clause 18, wherein the control line extends from the control cannula and returns to the control cannula from the second enlarged end of the tether, so that a free end of the control line is within the control cannula.

Structures or other features specified in the above clauses may be included singly or in any combination in the inventive devices, along with other structures or features described above with respect to any embodiment.

What is claimed is:

1. A closure for an opening in tissue, comprising:
a first closure element, the first closure element having a first mesh enclosure, the first mesh enclosure having a first distal narrowed end and a second proximal narrowed end and a central volume, each of the first and second ends being inverted so as to be within the central volume of the first mesh enclosure, each of the first and second ends being surrounded by respective external surfaces of the first mesh enclosure;
a second closure element, the second closure element having a second mesh enclosure physically separate from the first mesh enclosure, the second mesh enclosure having a third distal narrowed end and a fourth proximal narrowed end and a central volume, the third end being inverted so as to be within the central volume of the second mesh enclosure, each of the third and fourth ends being surrounded by respective external surfaces of the second mesh enclosure;
a tether joining the first and second closure elements in an initial configuration prior to delivery of the closure elements to the opening, the tether having first and second enlarged ends, wherein the tether extends through at least the first narrowed end, parallel to and alongside the second narrowed end and into the second closure element, so that the first enlarged end of the tether is outside the first closure element adjacent or engaging the first end and the second enlarged end of the tether is within the central volume of the second closure element and positioned to pass through the fourth narrowed end,
wherein the first closure element is adapted to engage a distal side of the tissue having the opening, and the second closure element is adapted to engage a proximal side of the tissue, and the tether is adapted to pass through the opening.

2. The closure of claim 1, further comprising a sheet of bioresorbable material fixed to the first closure element adjacent or over the second narrowed end.

3. The closure of claim 2, wherein the sheet is fixed to an external portion of the first mesh enclosure.

4. The closure of claim 2, wherein the sheet is fixed to an internal portion of the first mesh enclosure.

5. The closure of claim 1, further comprising a sheet of bioresorbable material fixed to the second closure element adjacent or over the third narrowed end.

6. The closure of claim 1, wherein each of the enlarged ends of the tether comprise a bead or a knot.

7. The closure of claim 1, further comprising a control line looped through the second enlarged end of the tether.

8. The closure of claim 7, wherein the control line passes through the fourth narrowed end.

9. The closure of claim 1, fitted within a delivery device.

10. The closure of claim 1, wherein the first and second narrowed ends are aligned with each other.

11. The closure of claim 1, wherein the third and fourth narrowed ends are aligned with each other.

12. The closure of claim 1, wherein the first and second narrowed ends are laterally offset with respect to each other.

13. The closure of claim 1, wherein one of the first and second narrowed ends is larger in diameter than the other of the first and second narrowed ends.

14. The closure of claim 1, wherein at least one of the first and second closure elements are adapted to expand in width during placement as at least one of the enlarged ends of the tether and at least one of the narrowed ends of at least one of the closure elements move with respect to each other.

15. A device for closing an opening in tissue, comprising:
a delivery device having a first peel-away catheter, a second tube within the first peel-away catheter, and a pusher tube within the second tube; and
the closure device of claim 1 fitted within the delivery device.

16. The device of claim 15, wherein the first closure element is within the first peel-away catheter and the second closure element is within the second tube.

17. The device of claim 15, further comprising a control line looped through the second enlarged end of the tether.

18. The device of claim 17, further comprising a control cannula extending through the pusher tube, and wherein the control line extends through the control cannula.

19. The device of claim 18, wherein the control line extends from the control cannula and returns to the control cannula from the second enlarged end of the tether, so that a free end of the control line is within the control cannula.
